(19) United States
(12) Patent Application Publication
(10) Pub. No.: US 2006/0233839 A1
(43) Pub. Date: Oct. 19, 2006

(54) Novel compounds

(76) Inventor: Alain Jacquet, Gosselies (BE)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/547,206

(22) PCT Filed: Feb. 24, 2004
(86) PCT No.: PCT/EP04/01850

§ 371 Date: May 19, 2006

(30) Foreign Application Priority Data
Feb. 26, 2003 (GB) 0304424.5

(51) Int. Cl. A61K 39/00; C07H 21/04; C12N 5/06; A61K 39/35; C07K 14/435; A61K 48/00

(52) U.S. Cl. 424/275.1; 514/44; 435/69.3; 435/320.1; 435/348; 530/350; 536/23.5

(57) ABSTRACT

The present invention provides a novel treatment for allergy comprising the provision of a recombinant Der p 1/ProDer p 1/PreProDer p 1 allergen derivative, ProDer p 3 or a recombinant ProDer p 3/Der p 3/PreProDer p 3 allergen derivative with hypoallergenic activity. Pharmaceutical compositions comprising said mutant allergens which stimulate a Th1-type immune response in allergic or naïve individuals thereby reducing the potential for an allergic response upon contact with the wild-type allergen, are also provided.

The present invention relates to novel prophylactic and therapeutic formulations, said formulations being effective in the prevention and/or the reduction of allergic responses to specific allergens. Further this invention relates to hypoallergenic recombinant derivatives of the major protein allergen from Dermatophagoides pteronyssinus, allergen Der p 1 and its precursor form ProDer p 1. In particular the derivatives of the invention include physically modified Der p 1 or ProDer p 1 such as the thermally treated protein; genetically modified recombinant Der p 1 wherein one or more cysteine residues involved in disulphide bond formation have been mutated; recombinant ProDer p 1; genetically modified recombinant ProDer p 1 wherein one or more cysteine residues involved in disulphide bond formation have been mutated; recombinant PreProDer p 1; or genetically modified recombinant PreProDer p 1 wherein one or more cysteine residues involved in disulphide bond formation have been mutated. Methods are also described for expressing and purifying the Der p 1, ProDer p 1 and PreProDer p 1 derivatives and for formulating immunogenic compositions and vaccines.

Further this invention relates to hypoallergenic recombinant derivatives of a further protein allergen from Dermatophagoides pteronyssinus, allergen Der p 3 and its precursor forms ProDer p 3 and PreProDer p 3. In particular the derivatives of the invention include physically modified Der p 3 or ProDer p 3 such as the thermally treated protein; genetically modified recombinant Der p 3 wherein one or more cysteine residues involved in disulphide bond formation have been mutated; recombinant ProDer p 3; genetically modified recombinant ProDer p 3 wherein one or more cysteine residues involved in disulphide bond formation have been mutated; recombinant PreProDer p 3; or genetically modified recombinant PreProDer p 3 wherein one or more cysteine residues involved in disulphide bond formation have been mutated. Methods are also described for expressing and purifying the Der p 3, ProDer p 3 and PreProDer p 3 derivatives and for formulating immunogenic compositions and vaccines.

Allergic responses in humans are common, and may be triggered by a variety of allergens. Allergic individuals are sensitised to allergens, and are characterised by the presence of high levels of allergen specific IgE in the serum, and possess allergen specific T-cell populations which produce Th2-type cytokines (IL-4, IL-5, and IL-13). Binding of IgE, in the presence of allergen, to FcεRI receptors present on the surface of mastocytes and basophils, leads to the rapid degranulation of the cells and the subsequent release of histamine, and other preformed and neoformed mediators of the inflammatory reaction. In addition to this, the stimulation of the T-cell recall response results in the production of IL-4 and IL-13, together cooperating to switch B-cell responses further towards allergen specific IgE production. For details of the generation of early and late phase allergic responses see Joost Van Neeven et al., 1996, Immunology Today, 17, 526. In non-allergic individuals, the immune response to the same antigens may additionally include Th1-type cytokines such as IFN-γ. These cytokines may prevent the onset of allergic responses by the inhibition of high levels of Th2-type immune responses, including high levels of allergen specific IgE. Importantly in this respect, is the fact that IgE synthesis may be controlled by an inhibitory feedback mechanism mediated by the binding of IgE/allergen complexes to the CD23 (FcεRII) receptor on B-cells (Luo et al., J. Immunol., 1991, 146(7), 2122-9; Yu et al., 1994, Nature, 369(6483):753-6). In systems that lack cellular bound CD23, this inhibition of IgE synthesis does not occur.

Type I allergic diseases mediated by IgE against allergens such as bronchial asthma, atopic dermatitis and perrenial rhinitis affect more than 20% of the world's population. Current strategies in the treatment of such allergic responses include means to prevent the symptomatic effects of histamine release by anti-histamine treatments and/or local administration of anti-inflammatory corticosteroids. Other strategies which are under development include those which use the hosts immune system to prevent the degranulation of the mast cells, Stanworth et al., EP 0 477 231 B1. Other forms of immunotherapy have been described (Hoyne et al., J. Exp. Med., 1993, 178, 1783-1788; Holt et al., Lancet, 1994, 344, 456-458).

While immediate as well as late symptoms can be ameliorated by pharmalogical treatment, allergen-specific immunotherapy is the only curative approach to type I allergy. However, some problems related to this method remain to be solved. First, immunotherapy is currently performed with total allergen extracts which can be heterogeneous from batch to batch. Moreover, these allergen mixtures are not designed for an individual patient's profile and may contain unwanted toxic proteins. Second, the administration of native allergens at high doses can cause severe anaphylactic reactions and therefore the optimally efficient high dose of allergen for successful immunotherapy can often not be reached. The first problem has been addressed through alternative vaccination with better characterised and more reproducible recombinant allergens as compared to allergen extracts. The second problem, namely the risk of anaphylactic reactions induced by repeated injections of allergen extracts, can be minimised through the use of recombinant “hypoallergens”, whose the IgE reactivity was altered by deletions or mutagenesis (Akdis, C A and Blaser, K, Regulation of specific immune responses by chemical and structural modifications of allergens, Int. Arch. Allergy Immunol., 2000, 121, 261-269).

Formulations have been described for the treatment and prophylaxis of allergy, which provide means to down-regulate the production of IgE, as well as modifying the cell mediated response to the allergen, through a shift from a Th2 type to a Th1 type of response (as measured by the reduction of ratio of IL-4 : IFN-γ producing Der p 1 specific T-cells, or alternatively a reduction of the IL-5:IFN-γ ratio). This may for example be achieved through the use of recombinant allergens such as recDer p 1 with reduced enzymatic activity as described in WO 99/25823. However the immunogenicity of these recombinant allergens is thought to be similar to that of wild-type ProDer p 1 in terms of IgE synthesis induction.

Non-anaphylactic forms of allergens with reduced IgE-binding activity have been reported. Allergen engineering has allowed a reduction of IgE-binding capacities of the allergen proteins by site-directed mutagenesis of amino acid residues or deletions of certain amino acid sequences. In the same time, T-cell activating capacity is still conserved as T cell epitopes are maintained. This has been shown using several approaches for different allergens although with variable results. Examples have been published for the timothy grass pollen allergen Ph1 p 5b (Schramm G et al., 1999, J Immunol., 162, 2406-14), for the major house dust mite allergens Derf2 (Takai et al. 2000, Eur. J. Biochem., 267, 6650-6656), DerP2 (Smith & Chapman 1996, Mol. Immunol. 33, 399-405) and Derf1 (Takahashi K et al. 2001, Int Arch Allergy Immunol. 124, 454-60). One study has reported the generation of Derf1 hypoallergens by introductions of point mutations at the level of cysteine residues involved in disulfides bridges (Takahashi K Int Arch Allergy Immunol. 2001;124(4):454-60., Takai T, Yasuhara T, Yokota T, Okumura Y). However, if wild-type ProDerf1 was successfully secreted by P. pastoris, cysteine mutants concerning intramolecular disulfide bonds were, by contrast, not secreted.

Allergens from the house dust mite Dermatophlagoides pteronyssinus are one of the major causative factors associated with allergic hypersensitivity reactions.

Der p 1

The group 1 allergen of Dermatophagoides pteronyssinus, Der p 1, is a major allergen, binding IgE in 80-100% of dust mite allergic sera (Chapman, M. D., et al. (1983). J. Allergy Clin. Immunol., 72: 27-33; Krillis, S., et al. (1984). J. Allergy Clin. Immunol., 74: 132-41). This protein is frequently found in high concentrations in house dust: from 100 to 10000 ng/g of dust (Platts-Mills and Chapman (1987). J. Allergy Clin. Immunol., 80: 755-75; Wahn, U., et al. (1997). J. Allergy Clin. Immunol., 99: 763-69), but Der p 1 is thought to be associated with a range of particles and not just faecal material (DeLuca, et al. (1999). J. Allergy Clin. Immunol., 103: 174-75). Levels of 100 ng are associated with sensitization and the risk increases with increasing doses.

The cDNA coding for Der p 1 has been cloned and sequenced (Chua, K., et al. (1988). J. Exp. Med., 167: 175-82; Thomas, et al. (1988). Int. Arch. Allergy Appl. Immunol., 85: 127-29; Chua, K., et al (1993). Int. Arch. Allergy Immunol., 101: 364-8): this allergen is a protein of 222 amino acid residues with a calculated molecular weight of 25 KDa. It is synthesized in a precursor form of 320 amino acid residues, including a 18-amino acid signal peptide and 80-amino acid N-terminal prosequence. The maturation process of ProDer p 1 is not known to date, but it is thought that the enzyme is activated by proteolytic removal of the pro region, or via autocatalytic processing.

The Der p 1 sequence displayed 30% homologies with that of papain, the cysteine proteinase archetype (Robinson, C., et al. (1997). Clin. Exp. Allergy, 27 (1): 10-21). Most of the residues implicated in the proteolytic activity of papain were conserved in Der p 1, including the cysteine and histidine residues of the active site. Due to the low availability of Der p1, no radiocrystallographic data has been obtained about this allergen. Nevertheless, the spatial structure of Der p 1 has been established based on the radiocrystallographic structure of papain and actinidin. The Der p 1 structure shares essential structural and mechanistic features with other papain-like cysteine proteinases. Below is a Der p 1 spatial structure model (Topham, C. M, et al. (1994) Protein engineering, 7 (7): 869-894). Der p 1 presents two globular domains formed independently by the N- and C-terminal sequences: The substrate binding and catalytic residues are in the cleft between the domains, and domains are connected by a flexible outside loop.

[image]

Although the cysteine protease activity of Der p 1 is generally accepted, studies have revealed that it exhibits a unique mixed cystein/serine protease activity, even though it has only one active site (Hewitt, C. R. A., et al (1997). Clin. Exp. Allergy, 27: 201-207). The preferred cleavage site is glutamate for the cysteine protease activity and arginine for the serine protease activity.

Der p 1 increases the permeability of bronchial mucosa, notably by degrading α1-antitrypsin, a protease inhibitor which protects these tissues (Kalsheker, et al. (1996). Biochem. Biophys. Res. Comm., 221: 59-61), and by loosening tight junctions in the respiratory epithelium (Wan, H., et al (2000). Clin. Exp. Allergy, 30:685-98), consequently facilitating access to antigen presenting cells. As shown in the scheme below, Der p 1 loosens tight junctions by cleavage of the protein “occluding”, facilitating absorption by dendritic cells and inducing allergic responses.

[image]

Der p 1 was shown to cleave CD23 (FcεR II), the low affinity 1gB receptor (Hewitt. C., et al (1995). J. Exp. Med., 182: 1537-1544; Schulz, O., et al. (1997). Eur. J. Immunol., 27: 584-588) involved in the regulation of IgE synthesis, thus stimulating IgE production. On the other hand it cleaves CD25, the α subunit of the IL-2 receptor (Schulz, O., et al (1998). J. Exp. Med., 187: 271-275). As IL-2 is a cytokine involved in the propagation of a Th1 immune response, the digestion of its receptor results in skewing towards a Th2 response. Proteolytic activity of Der p 1 has also been shown to enhance Th2 cytokine release from human T cells (Ghaemmaghami, A. M., et al. (2001). Eur. J. Immunol., 31: 1211-1216), and allow an adjuvant activity for a bystander allergen (Ghough L., et al. (2001). Clin. Exp Allergy, 31: 1594-1598).

Der p3

Der p 3 is a “group 3” allergen of Dermatophagoides pteronyssinus. Although generally considered a major allergen, estimates of Der p 3 IgE binding vary considerably, with frequencies as low as 16% (Heymann, P. W., et al (1989). J Allergy Clin Immunol., 83: 1055-1067) and as high as 100% with a potency similar to group 1 and 2 allergens (Stewart, G. A., et al. (1992). Immunology, 75: 29-35).

The cDNA coding for Der p 3 has been cloned and sequenced (Smith W. A., et al (1994). Clin. Exp Allergy, 24: 220-228): it is a protein of 232 amino acid residues with a calculated molecular weight of 25 KDa. The protein is synthesised as a inactive PreProDer p 3 percursor, with a 18-amino acid signal peptide, and a 11-amino acid N-terminal prosequence. Der p 3 is a serine proteinase displaying high homology with trypsin, the serine proteinase archetype (Stewart, G. A., et al. (1992). Immunology, 75: 29-35), including residues involved in the active site (FIG. 4.9). The preferred cleavage sites are arginine and lysine.

The Sequence homology between Der p 3 and bovine trypsin are shown below. Residues signalled with * are implicated in the catalytic site.

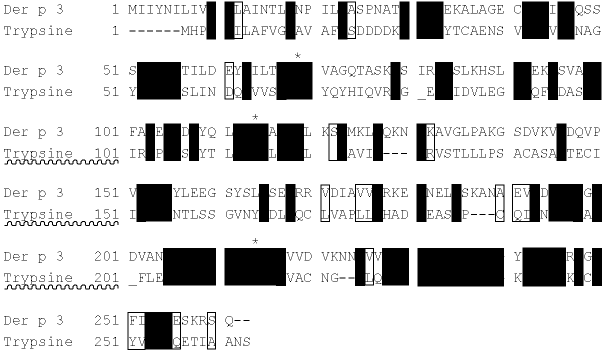

Der p 3 has been shown to trigger a signalling pathway, for the pro-inflammatory cytokines GM-CSF and eotaxin, by the activation of protease-activated receptor-2 on lung epithelial cells (Sum, G., et al. (2001). J. Immunol., 167: 1014-1021). In fact, it can further loosen tight junctions in the respiratory epithelium by cleaving the transmembrane protein occludin (Wan, H. et al. (2000). Clin. Exp. Allergy, 31: 279-294). This feature, also observed in Der p 1, provides a privileged access to antigen presenting cells.

The present invention relates to the provision and use of recombinant derivatives of Dermatophagoides pteronyssinus Der p 1 allergen or of its precursor forms ProDer p 1/preProDer p 1 thereafter referred to as “Der p 1/ProDer p 1/PreProDer p 1”, with reduced allergenic activity compared to the wild-type allergen. The recombinant forms of Der p 1 derivatives according to the invention, either adjuvanted recombinant proteins or plasmid encoding Der p 1/ProDer p 1/PreProDer p 1 suitable for NAVAC, are used as prophylactic or therapeutic vaccines to induce strong preventive Th1 or to shift Th2 to Th1 immune responses. The hypoallergenic derivatives can be successfully produced in recombinant expression systems and this is also an aspect of the present invention.

The present invention further relates to the provision and use of recombinant derivatives of Dermatophagoides pteronyssinus Der p 3 allergen or of its precursor forms ProDer p 3/preProDer p 3 thereafter referred to as “Der p 3/ProDer p 3/PreProDer p 3”, with reduced allergenic activity compared to the wild-type allergen. The recombinant forms of Der p 3 derivatives according to the invention, either adjuvanted recombinant proteins or plasmid encoding Der p 3/ProDer p 3/PreProDer p 3 suitable for NAVAC, are used as prophylactic or therapeutic vaccines to induce strong preventive Th1 or to shift Th2 to Th1 immune responses. The hypoallergenic derivatives can be successfully produced in recombinant expression systems and this is also an aspect of the present invention.

The present invention further relates to the provision and use of any combination of one or more protein allergens or recombinant derivatives as described herein. In one aspect, the present invention comprises or consists of Der p 1 or a derivative thereof as described herein, in combination with or fused with Der p 3, ProDer p 3 or ProDer p 1 or a derivative thereof as described herein. Preferably, the present invention provides Der p 1 in combination with or fused with ProDer p 3. In a yet further aspect, the present invention comprises or consists of Der p 3 or a derivative thereof as described herein, in combination with or fused with Der p 1, ProDer p 3, PreProDer p 3 or ProDer p 1, or PreProDer p 1 or a derivative thereof as described herein. In an alternative aspect, the present invention provides a fusion protein comprising ProDer p 3 fused with Der p 1. Any fusion protein as described herein may additionally comprise a series of histidines, preferably 6 histidines. In one aspect, the fusion protein comprises or consists of (His)₆-ProDer p 3-Der p 1.

Der p 1 is a 30 KDa protein and has been cloned and sequenced (Chua et al., 1988, J. Exp. Med., 167, 175-182). It is known to contain 222 amino acid residues in the mature protein. The sequence of Der p 1 shares 31% homology to papain, and shares more particularly homology in the enzymatically active regions, most notably the Cys34-His170 ion pair (Topham et al., supra). Der p 1 is produced in the mid-gut of the mite, where its role is probably related to the digestion of food. Up to 0.2 ng or proteolytically active Der p 1 is incorporated into each fecal pellet, each around 10-40 μm in diameter and, therefore, easily inspired into the human respiratory tract. Overnight storage of purified Der p 1 preparations at room temperature results in almost complete loss of enzymatic activity due to autoproteolytic degradation (Machado et al., 1996, Eur. J. Immunol. 26, 2972-2980). The Der p 1 encoding cDNA sequence reveals that, like many mammalian and plant proteinases, Der p 1 is synthetised as an inactive preproenzyme of 320 amino acid residues which is subsequently processed into a 222-amino acid mature form (Chua et al., 1988, J. Exp. Med., 167, 175-182; Chua et al., 1993, Int. Arch Allergy Immunol 101, 364-368). The maturation of ProDer p 1 is not known to date but it is thought that the allergen is processed by the cleavage of the 80-residues proregion.

The present invention provides a recombinant Dermatophagoides pteronyssinus Der p 1/ProDer p 1/ PreProDer p 1 protein allergen derivative wherein said allergen derivative has a significantly reduced allergenic activity compared to that the wild-type allergen. The allergenic activity can be impaired by several means which aim at disrupting the 3D-conformational shape of the protein forms by disrupting its intramolecular disulphide bridges thereby destabilising its 3-dimensional structure or by deleting a region of the protein, such as the amino acids 227-240 of ProDer p 1 (147-160 of the Der p 1 sequence). Said allergen derivatives having the following advantages over the unaltered wild-type allergen: 1) increases the Th1-type aspect of the immune responses (higher IgG2a for example) in comparison to those stimulated by the wild type allergen, thereby leading to the suppression of allergic potential of the vaccinated host, 2) having reduced allergenicity while still retaining T cell reactivity, thus being more suitable for systemic administration of high doses of the immunogen, 3) will induce Der p 1 specific IgG which compete with IgE for the binding of native Der p 1, 4) efficiently protects against airway eosinophilia even after exposure to aerosolised allergen extract. Such derivatives are suitable for use in therapeutic and prophylactic vaccine formulations which are suitable for use in medecine and more particularly for the treatment or prevention of allergic reactions.

According to a first aspect, the present invention provides a recombinant Der p 1/ProDer p 1/ PreProDer p 1 (i.e. Der p 1, ProDer p 1 or PreProDer p) allergen derivative wherein the allergenic activity has been significantly reduced, e.g. almost or completely abolished, by a physical means such as by thermally treating the protein, preferably in the presence of a reducing agent. Typically, the Der p 1/ProDer p 1/PreProDer p 1 protein is treated during a few minutes at about 100° C. in the presence of a reducing agent. Preferably the reducing agent is beta-mercaptoethanol or DTT. Still more preferably the protein is treated during 5 minutes at about 100° C. in the presence of 50 mM beta-mercaptoethanol. This treatment has a detrimental effect on the stability of the protein conformational IgE-binding epitopes. Preferably, the protein is ProDer p 1 or PreProDer p 1.

In a second aspect the present invention provides a recombinant Der p 1/ProDer p 1/PreProDer p 1 protein derivative wherein the allergenic activity has been genetically impaired such as by introducing specific mutations into the encoding cDNA or the genomic DNA. Accordingly an aspect of the invention provides the genetically mutated recombinant Der p 1/ProDer p 1/PreProDer p 1 per se. The reduction of the allergenicity of Der p 1/ProDer p 1/PreProDer p 1 may be performed by introducing mutations into the native sequence before recombinantly producing the hypoallergenic mutants. This may be achieved by: introducing substitutions, deletions, or additions in or by altering the three dimensional structure of the protein such that the tridimensional conformation of the protein is lost. This may be achieved, amongst others, by expressing the protein in fragments, or by deleting cysteine residues involved in disulphide bridge formation, or by deleting or adding residues such that the tertiary structure of the protein is substantially altered. Preferably, mutations may be generated with the effect of altering the interaction between two cysteine residues, typically one mutation at positions 4, 31, 65, 71, 103 and 117 of the native—mature—Der p 1 (which corresponds to positions 84, 111, 145, 151, 183 and 197 of ProDer p 1, respectively). A mutated protein according to the invention may comprise two or more (3, 4, 5 or all 6) cysteine mutations, thereby affecting different disulphide bridges, such as mutations at positions 4 & 31, 4 & 65, 4 & 71, 4 & 103, 31 & 65, or 4 & 31 & 65, or at positions 71 & 103, 71 & 117, 103 & 117, 31 &117,65& 117, or 71 & 103 & 117. Preferably the derivatives comprise one single mutation at any of the above positions. The most preferred mutation involves Cys4 (or alternatively, or in addition, Cys117 which is thought to be the disulphide bond partner of Cys4). The Cys mutations can be deletions, but are preferably substitutions for any of the other natural 19 amino acids. Preferred substitutions introduce positively charged amino acid residues to further destabilise the 3D-structure of the resulting protein. For example, preferred substitutions involve cysteine→arginine (or lysine) substitution.

In one aspect of the present invention, the derivatives comprise a triple mutation in which the cysteine residues 71, 103 and 117 are all mutated into alanine.

In a further aspect the present invention provides a form of ProDer p 1 in which the amino acids 227-240 of the ProDer p 1 sequence are deleted. These amino acids correspond to 147-160 of the Der p 1 sequence.

Accordingly, the invention is illustrated herein by, but is not limited to, six specific mutations which are given as examples of hypoallergenic Der p 1/ProDer p 1/PreProDer p 1 derivatives and a further mutation in which amino acids 227-240 of ProDer p 1 (147-160 of Der p 1) are deleted. First the allergenic activity of ProDer p 1 is substantially reduced, preferably completely abrogated by substituting a cysteine residue for an arginine residue at position Cys4 of Der p 1 protein sequence, and is set out in SEQ ID NO:3. Second, the allergenic activity of ProDer p 1 is substantially abrogated by substituting a cysteine residue for an arginine residue at any of the following positions (calculated by reference to the sequence in mature Der p 1): Cys31 of Der p 1 protein sequence (SEQ ID NO:5), Cys65 ( SEQ ID NO:7), Cys71 (SEQ ID NO:9), Cys103 (SEQ ID NO:11), Cys117 (SEQ ID NO:13).

Further, the allergenic activity of ProDer p 1 is substantially reduced, preferably completely abrogated, by deletion of the amino acids 227-240 of ProDer p 1 (147-160 of Der p 1) (SEQ ID NO:15).

Mutated versions of Der p 1/ProDer p 1/PreProDer p 1 may be prepared by site-directed mutagenesis of the cDNA which codes for the Der p 1/ProDer p 1/PreProDer p 1 protein by conventional methods such as those described by G. Winter et al in Nature 1982, 299, 756-758 or by Zoller and Smith 1982; Nucl. Acids Res., 10, 6487-6500, or deletion mutagenesis such as described by Chan and Smith in Nucl. Acids Res., 1984, 12, 2407-2419 or by G. Winter et al in Biochem. Soc. Trans., 1984, 12, 224-225.

Hypoallergenic ProDer p 1, PreProDer p 1, Der p 3, ProDer p 3 or PreProDer p 3 is also provided by the present invention.

The invention is not limited to the specifically disclosed sequence, but includes any hypoallergenic allergen which has been mutated to decrease or abolish its IgE-binding reactivity and/or histamine release activity, whilst retaining its T cell reactivity and/or the ability to stimulate an immune response against the wild-type allergen. The allergenic activity, and consequently the reduction in the allergenic activity, of the mutant allergens may be compared to the wild type by any of the following methods: histamine release activity or by IgE-binding reactivity, according to the method detailed in the Example section.

“Substantially reduced allergenic activity” means that the allergenic activity as measured by residual IgE-binding activity is reduced to a maximum of 50% of the activity of the native—unmodified or unmutated—protein, preferably to a maximum of 20%, more preferably to a maximum of 10%, still more preferably to a maximum of 5%, still more preferably to less than 5%. Alternatively, “substantially reduced allergenic activity” can also be assessed by measuring the histamine release activity of the mutant. A substantial reduction in activity is when there is a reduction of at least a 100-fold factor as compared to the native protein, preferably by a factor of 1000-fold, still more preferably by a factor of 10000-fold.

The immunogenicity of the mutant allergen may be compared to that of the wild-type allergen by various immunologicals assays. The cross-reactivity of the mutant and wild-type allergens may be assayed by in vitro T-cell assays after vaccination with either mutant or wild-type allergens. Briefly, splenic T-cells isolated from vaccinated animals may be restimulated in vitro with either mutant or wild-type allergen followed by measurement of cytokine production with commercially available ELISA assays, or proliferation of allergen specific T cells may be assayed over time by incorporation of tritiated thymidine. Also the immunogenicity may be determined by ELISA assay, the details of which may be easily determined by the man skilled in the art. Briefly, two types of ELISA assay are envisaged. First, to assess the recognition of the mutant Der p 1 by sera of mice immunized with the wild type Der p 1; and secondly by recognition of wild type Der p 1 allergen by the sera of animals immunised with the mutant allergen. Briefly, each wells will be coated with 500 ng of purified wild type or mutated Der p 1 overnight at 4° C. After incubating with a blocking solution (TBS-Tween 0.1% with 1% BSA) successive dilutions of sera will be incubated at 37° C. for 1 hour. The wells are washed 5 times, and total IgG revealed by incubating with an anti-IgG antibody conjugated with Alkaline phosphatase. The immunogenicity of mutant Der p 3 may be compared to wild-type Der p 3 as described for Der p 1, above.

A further aspect of the present invention provides an isolated nucleic acid encoding a mutated version of the Der p 1/ProDer p 1/PreProDer p 1 allergen as disclosed herein. Preferably the nucleotide sequence is a DNA sequence and can be synthesized by standard DNA synthesis techniques, such as by enzymatic ligation as described by D. M. Roberts et al in Biochemistry 1985, 24, 5090-5098, by chemical synthesis, by in vitro enzymatic polymerization, or by a combination of these techniques. Preferably the nucleic acid sequence has a codon usage pattern that has been optimised so as to mimic the one used in the intended expression host, more preferably resembling that of highly expressed mammalian e.g. human genes. Preferred DNA sequences are codon-optimised sequences and are set out in SEQ ID NO:4, SEQ ID NO:6, SEQ ID NO:8, SEQ ID NO:10, SEQ ID NO:12, SEQ ID NO:14, SEQ ID NO:15 and SEQ ID NO:17.

A further aspect of the present invention provides an isolated nucleic acid encoding a mutated version of the Der p 3/ProDer p 3/PreProDer p 3 allergen as disclosed herein. Preferably the nucleotide sequence is a DNA sequence and can be synthesized by standard DNA synthesis techniques, such as by enzymatic ligation as described by D. M. Roberts et al in Biochemistry 1985, 24, 5090-5098, by chemical synthesis, by in vitro enzymatic polymerization, or by a combination of these techniques. Preferably the nucleic acid sequence has a codon usage pattern that has been optimised so as to mimic the one used in the intended expression host, more preferably resembling that of highly expressed mammalian e.g. human genes. A preferred DNA sequence is set out in SEQ ID NOs:20 and 21.

Enzymatic polymerisation of DNA may be carried out in vitro using a DNA polymerase such as DNA polymerase I (Klenow fragment) in an appropriate buffer containing the nucleoside triphosphates dATP, dCTP, dGTP and dTTP as required at a temperature of 10°-37° C., generally in a volume of 50 ml or less. Enzymatic ligation of DNA fragments may be carried out using a DNA ligase such as T4 DNA ligase in an appropriate buffer, such as 0.05M Tris (pH 7.4), 0.01M MgCl₂, 0.01M dithiothreitol, 1 mM spermidine, 1 mM ATP and 0.1 mg/ml bovine serum albumin, at a temperature of 4° C. to ambient, generally in a volume of 50 ml or less. The chemical synthesis of the DNA polymer or fragments may be carried out by conventional phosphotriester, phosphite or phosphoramidite chemistry, using solid phase techniques such as those described in ‘Chemical and Enzymatic Synthesis of Gene Fragments—A Laboratory Manual’ (ed. H. G. Gassen and A. Lang), Verlag Chemie, Weinheim (1982),or in other scientific publications, for example M. J. Gait, H. W. D. Matthes, M. Singh, B. S. Sproat, and R. C. Titmas, Nucleic Acids Research, 1982, 10, 6243; B. S. Sproat and W. Bannwarth, Tetrahedron Letters, 1983, 24, 5771; M. D. Matteucci and M. H Caruthers, Tetrahedron Letters, 1980, 21, 719; M. D. Matteucci and M. H. Caruthers, Journal of the American Chemical Society, 1981, 103, 3185; S. P. Adams et al., Journal of the American Chemical Society, 1983, 105, 661; N. D. Sinha, J. Biernat, J. McMannus, and H. Koester, Nucleic Acids Research, 1984, 12, 4539; and H. W. D. Matthes et al., EMBO Journal, 1984, 3, 801.

Alternatively, the coding sequence can be derived from Der p 1/ProDer p 1/PreProDer p 1 mRNA, using known techniques (e.g. reverse transcription of mRNA to generate a complementary cDNA strand), and commercially available cDNA kits. The coding sequence of Der p 3/ProDer p 3/PreProDer p 3 may be derived as described above; the codon usage pattern of the PreProDer p 3 nucleotide sequence is typical of highly expressed bacterial genes.

Surprisingly, it has been found that ProDer p 3 is highly hypoallergenic compared to Der p 3.

Desirably the codon usage pattern of the nucleotide sequence is typical of highly expressed human genes. Accordingly there is provided in a particular aspect of the invention a nucleotide sequence comprising a plurality of codons together encoding the mutated Der p 1/ProDer p 1/PreProDer p 1 protein, wherein the selection of the possible codons used for encoding the recombinant mite protein amino acid sequence has been changed to closely mimic the optimised mammalian codon usage, such that the frequency of codon usage in the resulting gene sequence is substantially the same as a mammalian gene which would encode the same protein. Codon usage patterns for mammals, including humans, can be found in the literature (see e.g. Nakamura et al. 1996, Nucleic Acids Res. 24, 214-215.

The DNA code has 4 letters (A, T, C and G) and uses these to spell three letter “codons” which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons.

Where more than one codon is available to code for a given amino acid, it has been observed that the codon usage patterns of organisms are highly non-random. Different species show a different bias in their codon selection and, furthermore, utilization of codons may be markedly different in a single species between genes which are expressed at high and low levels. This bias is different in viruses, plants, bacteria, insect and mammalian cells, and some species show a stronger bias away from a random codon selection than others. For example, humans and other mammals are less strongly biased than certain bacteria or viruses. For these reasons, there is a significant probability that a mammalian gene expressed in E.coli or a viral gene expressed in mammalian cells will have an inappropriate distribution of codons for efficient expression. However, a gene with a codon usage pattern suitable for E.coli expression may also be efficiently expressed in humans. It is believed that the presence in a heterologous DNA sequence of clusters of codons which are rarely observed in the host in which expression is to occur, is predictive of low heterologous expression levels in that host.

There are several examples where changing codons from those which are rare in the host to those which are host-preferred (“codon optimisation”) has enhanced heterologous expression levels, for example the BPV (bovine papilloma virus) late genes L1 and L2 have been codon optimised for mammalian codon usage patterns and this has been shown to give increased expression levels over the wild-type HPV sequences in mammalian (Cos-1) cell culture (Zhou et. al. J. Virol 1999. 73, 4972-4982). In this work, every BPV codon which occurred more than twice as frequently in BPV than in mammals (ratio of usage >2), and most codons with a usage ratio of >1.5 were conservatively replaced by the preferentially used mammalian codon. In WO97/31115, WO97/48370 and WO98/34640 (Merck & Co., Inc.) codon optimisation of HIV genes or segments thereof has been shown to result in increased protein expression and improved immunogenicity when the codon optimised sequences are used as DNA vaccines in the host mammal for which the optimisation was tailored.

In this work, the sequences preferably consist entirely of optimised codons (except where this would introduce an undesired restriction site, intron splice site etc.) because each D. pteronyssinus codon is conservatively replaced with the optimal codon for a mammalian host. Surprisingly such optimised ProDer p 1/Der p 1 sequences also express very well in yeast despite the different codon usage of yeast.

A still further aspect of the invention provides a process for the preparation of a mutated Der p 1/ProDer p 1/ PreProDer p 1 protein which process comprises expressing DNA, either codon optimised or not, encoding the said protein in a recombinant host cell and recovering the product; the above process also applies for Der p 3/ProDer p 3/PreProDer p 3.

Although Der p 1 is well characterized in terms of its enzymatic activity, allergenicity and gene cloning, heterologous expression of Der p 1 has been reported to be problematic (Chapman and Platts-Mills, J Immunol 1980;125:587-592), probably because this cysteine proteinase is synthesized as a PreProDer p 1 precursor. Even more problematic is the expression of Der p 1/ProDer p 1/PreProDer p 1 sequences wherein cysteine residues involved in the protein conformation have been mutated. Accordingly the present invention further provides a process overcoming all these drawbacks therefore allowing the production of the mutated proteins and the industrial development of therapeutic and prophylactic vaccines to mite allergy.

A process for production of Der p 3/ProDer p 3/PreProDer p 3 mutated or recombinant proteins is also provided.

A substantial amelioration of protein expression has been achieved in E. coli when Der p 1/ProDer p 1/PreProDer p 1 either mutated or not was expressed as a Maltose Binding Protein (MBP) fusion protein. Accordingly there is provided a process for expressing the mutated ProDerP/Der p 1 protein as a MBP fusion protein in E. coli. Furthermore, a substantial amelioration of protein expression in yeast has been surprisingly achieved for the mutated protein even though disulphide bonds are said to be essential for secretion in Pichia pastoris (Takai et al. 2001, Int. Arch. Allergy Immunol. 124, 454-460). This was achieved by re-engineering the polynucleotide sequence which encodes the Dermaphagoides mutated ProDerP/Der p 1 protein to fit the codon usage found in highly expressed human genes, thereby also allowing the recombinant antigen to have the same conformation and immunological properties as native ProDerP/Der p 1 Dermaphagoides allergens. Surprisingly, the cloning and expression of mutated ProDer p 1, codon-optimised for mammalian cell expression, could be achieved in Pichia pastoris, with a certain proportion being secreted, although expression in P. pastoris has been formerly reported to be unsuccessful (Takai et al. 2001, Int. Arch. Allergy Immunol. 124, 454-460).

The process of the invention may be performed by conventional recombinant techniques such as described in Maniatis et. al., Molecular Cloning—A Laboratory Manual; Cold Spring Harbor, 1982-1989.

In particular, the process may comprise the steps of:
1. Preparing a replicable or integrating expression vector capable, in a host cell, of expressing a DNA polymer comprising a nucleotide sequence that encodes the said Der p 1/ProDer p 1/PreProDer p 1 protein;
2. Altering the IgE-binding activity of the resultant protein by replacing the cysteine residues involved in disuphide bonds with another residue, preferably an arginine residue, using site directed mutagenesis;
3. Transforming a host cell with the said vector
4. Culturing the transformed host cell under conditions permitting expression of the DNA polymer to produce the protein; and
5. Recovering the protein.

The above process may also apply for Der p 3/ProDer p 3/PreProDer p 3

The term ‘transforming’ is used herein to mean the introduction of foreign DNA into a host cell by transformation, transfection or infection with an appropriate plasmid or viral vector using e.g. conventional techniques as described in Genetic Engineering; Eds. S. M. Kingsman and A. J. Kingsman; Blackwell Scientific Publications; Oxford, England, 1988. The term ‘transformed’ or ‘transformant’ will hereafter apply to the resulting host cell containing and expressing the foreign gene of interest.

The expression vector is novel and also forms part of the invention. One particular aspect of the present invention provides an expression vector which comprises, and is capable of directing the expression of, a polynucleotide sequence encoding a cystein-mutated Der p 1/ProDer p 1/PreProDer p 1 protein according to the invention. Another particular aspect of the invention provides an expression vector which comprises, and is capable of directing the expression of, a polynucleotide sequence encoding a cysteine-mutated Der p 1/ProDer p 1/PreProDer p 1 protein wherein the codon usage pattern of the polynucleotide sequence is typical of highly expressed mammalian genes, preferably highly expressed human genes. The vector may be suitable for driving expression of heterologous DNA in bacterial, insect, yeast or mammalian cells, particularly human cells.

The replicable expression vector may be prepared in accordance with the invention, by cleaving a vector compatible with the host cell to provide a linear DNA segment having an intact replicon, and combining said linear segment with one or more DNA molecules which, together with said linear segment encode the desired product, such as the DNA polymer encoding the Der p 1/ProDer p 1/PreProDer p 1 protein under ligating conditions.

The above vectors may also apply for mutated Der p 3/ProDer p 3/PreProDer p 3 according to the present invention.

Thus, the DNA polymer may be preformed or formed during the construction of the vector, as desired.

The choice of vector will be determined in part by the host cell, which may be prokaryotic or eukaryotic. Suitable vectors include plasmids, bacteriophages, cosmids and recombinant viruses.

The preparation of the replicable expression vector may be carried out conventionally with appropriate enzymes for restriction, polymerisation and ligation of the DNA, by procedures described in, for example, Maniatis et al cited above. The recombinant host cell is prepared, in accordance with the invention, by transforming a host cell with a replicable expression vector of the invention under transforming conditions. Suitable transforming conditions are conventional and are described in, for example, Maniatis et al cited above, or “DNA Cloning” Vol. II, D. M. Glover ed., IRL Press Ltd, 1985.

The choice of transforming conditions is determined by the host cell. Thus, a bacterial host such as E. coli may be treated with a solution of CaCl₂ (Cohen et al, Proc. Nat. Acad. Sci., 1973, 69, 2110) or with a solution comprising a mixture of RbCl, MnCl₂, potassium acetate and glycerol, and then with 3-[N-morpholino]-propane-sulphonic acid, RbCl and glycerol. Mammalian cells in culture may be transformed by calcium co-precipitation of the vector DNA onto the cells, by lipofection, or by electroporation. Yeast compatible vectors also carry markers that allow the selection of successful transformants by conferring prototrophy to auxotrophic mutants or resistance to heavy metals on wild-type strains. Control sequences for yeast vectors include promoters for glycolytic enzymes (Hess et al., J. Adv. Enzyme Reg. 1968, 7, 149), PHO5 gene encoding acid phosphatase, CUP1 gene, ARG3 gene, GAL genes promoters and synthetic promoter sequences. Other control elements useful in yeast expression are terminators and leader sequences. The leader sequence is particularly useful since it typically encodes a signal peptide comprised of hydrophobic amino acids, which direct the secretion of the protein from the cell. Suitable signal sequences can be encoded by genes for secreted yeast proteins such as the yeast invertase gene and the a-factor gene, acid phosphatase, killer toxin, the a-mating factor gene and recently the heterologous inulinase signal sequence derived from INU1A gene of Kluyveromyces marxianus. Suitable vectors have been developed for expression in Pichia pastoris and Saccharomyces cerevisiae.

A variety of P. pastoris expression vectors are available based on various inducible or constitutive promoters (Cereghino and Cregg, FEMS Microbiol. Rev. 2000,24:45-66). For the production of cytosolic and secreted proteins,the most commonly used P. pastoris vectors contain the very strong and tightly regulated alcohol oxidase (AOX1) promoter. The vectors also contain the P. pastoris histidinol dehydrogenase (HIS4) gene for selection in his4 hosts. Secretion of foreign protein require the presence of a signal sequence and the S. cerevisiae prepro alpha mating factor leader sequence has been widly and successfully used in Pichia expression system. Expression vectors are integrated into the P. pastoris genome to maximize the stability of expression strains. As in S. cerevisiae, cleavage of a P. pastoris expression vector within a sequence shared by the host genome (AOX1 or HIS4) stimulates homologous recombination events that efficiently target integration of the vector to that genomic locus. In general, a recombinant strain that contains multiple integrated copies of an expression cassette can yield more heterologous protein than single-copy strain. The most effective way to obtain high copy number transformants requires the transformation of Pichia recipient strain by the sphaeroplast technique (Cregg et all 1985, Mol. Cell. Biol. 5: 3376-3385).

The invention also extends to a host cell transformed with a replicable expression vector of the invention.

Culturing the transformed host cell under conditions permitting expression of the DNA polymer is carried out conventionally, as described in, for example, Maniatis et al and “DNA Cloning” cited above. Thus, preferably the cell is supplied with nutrient and cultured at a temperature below 45° C.

The product is recovered by conventional methods according to the host cell. Thus, where the host cell is bacterial, such as E. coli it may be lysed physically, chemically or enzymatically and the protein product isolated from the resulting lysate. Where the host cell is mammalian, the product may generally be isolated from the nutrient medium or from cell free extracts. Conventional protein isolation techniques include selective precipitation, absorption chromatography, and affinity chromatography including a monoclonal antibody affinity column.

Alternatively, the expression may be carried out either in insect cells using a suitable vector such as a baculovirus, in transformed drosophila cells, or mammalian CHO cells. The novel protein of the invention may also be expressed in yeast cells as described for the CS protein in EP-A-0 278 941.

Pharmaceutical, immunogenic and vaccine compositions comprising a hypoallergenic Der p 1/ProDer p 1/PreProDer p 1 derivative according to the invention, or the polynucleotide sequences encoding said proteins, either codon-optimised or not, are also provided. Such compositions comprising hypoallergenic Der p 3/ProDer p 3/PreProDer p 3 are also provided. In preferred embodiments the DNA composition comprises a plurality of particles, preferably gold particles, coated with DNA comprising a vector encoding a polynucleotide sequence which encodes a D. pteronyssinus amino acid sequence, wherein the codon usage pattern of the polynucleotide sequence is typical of highly expressed mammalian genes, particularly human genes.

The polynucleotides and encoded polypeptides according to the invention may find use as therapeutic or prophylactic agents. In particlular the polynucleotides of the invention (including a polynucleotide sequence of native ProDer p 1—preferably codon optimised) may be used in DNA vaccination (NAVAC), the DNA being administered to the mammal e.g. human to be vaccinated. The nucleic acid, such as RNA or DNA, preferably DNA, is provided in the form of a vector, such as those described above, which may be expressed in the cells of the mammal. The polynucleotides may be administered by any available technique. For example, the nucleic acid may be introduced by needle injection, preferably intradermally, subcutaneously or intramuscularly. Alternatively, the nucleic acid may be delivered directly into the skin using a nucleic acid delivery device such as particle-mediated DNA delivery (PMDD). In this method, inert particles (such as gold beads) are coated with a nucleic acid, and are accelerated at speeds sufficient to enable them to penetrate a surface of a recipient (e.g. skin), for example by means of discharge under high pressure from a projecting device. (Particles coated with a nucleic acid molecule of the present invention are within the scope of the present invention, as are delivery devices loaded with such particles).

Suitable techniques for introducing the naked polynucleotide or vector into a patient include topical application with an appropriate vehicle. The nucleic acid may be administered topically to the skin, or to mucosal surfaces for example by intranasal, oral, intravaginal or intrarectal administration. The naked polynucleotide or vector may be present together with a pharmaceutically acceptable excipient, such as phosphate buffered saline (PBS). DNA uptake may be further facilitated by use of facilitating agents such as bupivacaine, either separately or included in the DNA formulation. Other methods of administering the nucleic acid directly to a recipient include ultrasound, electrical stimulation, electroporation and microseeding which is described in U.S. Pat. No. 5,697,901. Typically the nucleic acid is administered in an amount in the range of 1 pg to 1 mg, preferably 1 pg to 10 μg nucleic acid for particle mediated gene delivery and 10 μg to 1 mg for other routes.

A nucleic acid sequence of the present invention may also be administered by means of specialised delivery vectors useful in gene therapy. Gene therapy approaches are discussed for example by Verme et al, Nature 1997, 389:239-242. Both viral and non-viral vector systems can be used. Viral based systems include retroviral, lentiviral, adenoviral, adeno-associated viral, herpes viral, Canarypox and vaccinia-viral based systems. Non-viral based systems include direct administration of nucleic acids, microsphere encapsulation technology (poly(lactide-co-glycolide) and, liposome-based systems. Viral and non-viral delivery systems may be combined where it is desirable to provide booster injections after an initial vaccination, for example an initial “prime” DNA vaccination using a non-viral vector such as a plasmid followed by one or more “boost” vaccinations using a viral vector or non-viral based system.

In this way, the inventors have found that vaccination with DNA encoding ProDer p 1 (preferably codon optimised for mammals) induces a Th1 response in mice models (high titres of specific IgG2a antibodies and low titres of specific IgG1) and, remarkably, the absence of anti-ProDer p 1 IgE.

The pharmaceutical compositions of the present invention may include adjuvant compounds, or other substances which may serve to increase the immune response induced by the protein.

The vaccine composition of the invention comprises an immunoprotective amount of the mutated or recombinant version of the Der p 1/ProDer p 1/PreProDer p 1 hypoallergenic protein or the mutated or recombinant version of the Der p 3/ProDer p 3/PreProDer p 3 hypoallergenic protein. The term “immunoprotective” refers to the amount necessary to elicit an immune response against a subsequent challenge such that allergic disease is averted or mitigated. In the vaccine of the invention, an aqueous solution of the protein can be used directly. Alternatively, the protein, with or without prior lyophilization, can be mixed, adsorbed, or covalently linked with any of the various known adjuvants.

Suitable adjuvants are commercially available such as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleulin-2, -7, or -12, and chemokines may also be used as adjuvants.

In the formulations of the invention it is preferred that the adjuvant composition induces an immune response predominantly of the Th1 type. High levels of Th1-type cytokines (e.g., IFN-γ, TNFα, IL-2 and IL-12) tend to favour the induction of cell mediated immune responses to an administered antigen. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Accordingly, suitable adjuvants for use in eliciting a predominantly Th1-type response include, for example a combination of monophosphoryl lipid A, preferably 3-de-O-acylated monophosphoryl lipid A (3D-MPL) together with an aluminium salt. Other known adjuvants, which preferentially induce a TH1 type immune response, include CpG containing oligonucleotides. The oligonucleotides are characterised in that the CpG dinucleotide is unmethylated. Such oligonucleotides are well known and are described in, for example WO 96/02555. Immunostimulatory DNA sequences are also described, for example, by Sato et al., Science 273:352, 1996. CpG-containing oligonucleotides may also be used alone or in combination with other adjuvants. For example, an enhanced system involves the combination of a CpG-containing oligonucleotide and a saponin derivative particularly the combination of CpG and QS21 as disclosed in WO 00/09159 and WO 00/62800. Preferably the formulation additionally comprises an oil in water emulsion and/or tocopherol.

Another preferred adjuvant is a saponin, preferably QS21 (Aquila Biopharmaceuticals Inc., Framingham, Mass.), that may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

A particularly potent adjuvant formulation involving QS21 3D-MPL & tocopherol in an oil in water emulsion is described in WO 95/17210 and is a preferred formulation.

Other preferred adjuvants include Montanide ISA 720 (Seppic, France), SAF (Chiron, California, United States), ISCOMS (CSL), M-59 (Chiron), Detox (Ribi, Hamilton, Mont.), RC-529 (Corixa, Hamilton, Mont.) and other aminoalkyl glucosaminide 4-phosphates (AGPs).

Accordingly there is provided an immunogenic composition comprising a Der p 1/ProDer p 1/PreProDer p 1 hypoallergenic derivative as disclosed herein and an adjuvant, wherein the adjuvant comprises one or more of 3D-MPL, QS21, a CpG oligonucleotide, a polyethylene ether or ester or a combination of two or more of these adjuvants. The Der p 1/ProDer p 1/PreProDer p 1 hypoallergenic derivative within the immunogenic composition is preferably presented in an oil in water or a water in oil emulsion vehicle.

There is further provided an immunogenic composition comprising a Der p 3/ProDer p 3/PreProDer p 3 hypoallergenic derivative as disclosed herein and an adjuvant, wherein the adjuvant comprises one or more of 3D-MPL, QS21, a CpG oligonucleotide, a polyethylene ether or ester or a combination of two or more of these adjuvants. The Der p 3/ProDer p 3/ PreProDer p hypoallergenic derivative within the immunogenic composition is preferably presented in an oil in water or a water in oil emulsion vehicle.

In a further aspect, the present invention provides a method of making a pharmaceutical composition including the step of mutating one or more cysteine residues of Der p 1/ProDer p 1/PreProDer p 1/Der p 3/ProDer p 3/PreProDer p 3 involved in disulphide bridge formation, for example mutation of the following residues of Der p 1: Cys4, Cys31, Cys65, Cys71, Cys103 or Cys117. In an alternative aspect, the invention provides a method of making a pharmaceutical composition including the step of deleting the amino-acid residues 227-240 of ProDer p 1 (147-160 of Der p 1).

The method further comprises the step of altering the codon usage pattern of a wild-type Der p 1/ProDer p 1/PreProDer p 1 nucleotide sequence, or creating a polynucleotide sequence synthetically, to produce a sequence having a codon usage pattern typical of highly expressed mammalian genes and encoding a codon-optimised cysteine-mutated ProDer p 1/Der p 1 amino acid sequence or a ProDer p 1/Der p 1 amino-acid sequence in which selected residues have been deleted according to the invention. Vaccine preparation is generally described in Vaccine Design (“The subunit and adjuvant approach” (eds. Powell M. F. & Newman M. J). (1995) Plenum Press New York). Encapsulation within liposomes is described by Fullerton, U.S. Pat. No. 4,235,877. Conjugation of proteins to macromolecules is disclosed, for example, by Likhite, U.S. Pat. No. 4,372,945 and Armor et al., U.S. Pat. No. 4,474,757.

The amount of the protein of the present invention present in each vaccine dose is selected as an amount which induces an immunoprotective response without significant, adverse side effects in typical vaccines. Such amount will vary depending upon which specific immunogen is employed and whether or not the vaccine is adjuvanted. Generally, it is expected that each dose will comprise 1-1000 μg of protein, preferably 1-200 μg. An optimal amount for a particular vaccine can be ascertained by standard studies involving observation of antibody titres and other responses in subjects. The vaccines of the present invention may be administered to adults or infants, however, it is preferable to vaccinate individuals soon after birth before the establishment of substantial Th2-type memory responses. Following an initial vaccination, subjects will preferably receive a boost in about 4 weeks, followed by repeated boosts every six months for as long as a risk of allergic responses exists.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

The present invention also provides a process for the production of a vaccine, comprising the steps of purifying a Der p 1/ProDer p 1/PreProDer p 1 derivative or ProDer p 3/Der p 3/PreProDer p 3 derivative according to the invention or a derivative thereof, by the process disclosed herein and admixing the resulting protein with a suitable adjuvant, diluent or other pharmaceutically acceptable excipient.

The present invention also provides a method for producing a vaccine formulation comprising mixing a protein of the present invention together with a pharmaceutically acceptable excipient.

Another aspect of the invention is the use of a protein or polynucleotide as claimed herein for the manufacture of a vaccine for immunotherapeutically treating a patient susceptible to or suffering from allergy. A method of treating patients susceptible to or suffering from allergy comprising administering to said patients a pharmaceutically active amount of the immunogenic composition disclosed herein is also contemplated by the present invention.

A further aspect of the invention provides a method of preventing or mitigating an allergic disease in man (particularly house dust mite allergy), which method comprises administering to a subject in need thereof an immunogenically effective amount of a mutated allergen of the invention, or of a vaccine in accordance with the invention.

### FIGURE LEGENDS

FIG. 1: IgG and IgE-binding reactivity of denatured ProDer p 1 expressed in CHO cells. Immunoplates were coated with 500 ng/well of purified native or denatured ProDer p 1 and incubated with sera (diluted 1:8) radioallergosorbent positive to D. pteronyssinus. Bound IgE or IgG were quantitated by incubation with mouse anti-human IgE or IgG and alkaline phosphatase-labelled anti-mouse IgG antibodies, followed by an enzymatic assay. Results are expressed as OD₄₁₀ₙₘ values.

FIG. 2: Correlation between the IgE reactivity of MBP-ProDer p 1 and natural DerP. Immunoplates were coated with 500 ng/well of purified DerP or MBP-ProDer p 1 and inculated with 95 sera (diluted 1:8) radioallergosorbent positive to D. pteronyssinus. Bound IgE was quantitated by incubation with mouse anti-human IgE and alkaline phosphatase-labelled anti-mouse Ig antibodies, followed by an enzymatic assay. Results are expressed as OD₄₁₀ₙₘ values.

FIG. 3: IgE-binding reactivities of MBP-ProDer p 1 mutants, carrying the mutations C4R, C31R and C65R. Immunoplates were coated with 500 ng/well of Wild-type or mutant MBP-ProDer p 1 and incubated with a pool of 20 sera (diluted 1:8) radioallergosorbent positive to D. pteronyssinus. Bound IgE was quantitated by incubation with mouse anti-human IgE and alkaline phosphatase-labelled anti-mouse IgG antibodies, followed by an enzymatic assay. Results are expressed as OD₄₁₀ₙₘ values.

FIG. 4: Histamine release activity of allergens. Basophils isolated from the peripheral blood of one allergic donor were stimulated with serial dilutions of different allergens. The histamine released from cells was measured by ELISA. The total amount of histamine in basophils was quantified after cell disruption with the detergent IGEPAL CA-630. Results are shown as the ratio of released histamine by allergens to total histamine.

FIG. 5: schematic representation of the animal model of house dust mite allergy.

FIG. 6: expression of ProDer p 1 Δ227-240 in P. pastoris after induction with methanol for 24 and 48 hours. The culture supernatants of the recombinant clones are analysed by SDS PAGE. The blot is revealed by means of a polyclonal mouse serum against ProDer p 1 expressed in CHO. Tracks 1,2: irrelevant proteins, Track 3: yeasts not induced, Track 4: purified ProDer p 1, Track 5: clone 1 after induction for 24 hours, Track 6: clone 1 after induction for 48 hours, Track 7: clone 2 after induction for 24 hours, Track 8: clone 2 after induction for 48 hours.

FIG. 7

Sequence comparison of wild-type (AcaNucSeq) and bacterial codon-optimized (EcoNucSeq) PreProDer p 3 cDNA. The deduced amino acid sequence shown below each codon is designated by the single-letter code. The leader peptide and the propeptide sequence are indicated in italics and underlined respectively.

FIG. 8

Expression of ProDer p 3

After induction with 0.5 or 1 mM IPTG for 1, 2, 3 and 16 h, the bacteria were crushed. The cytoplasmic fraction (S) and the insoluble fraction (C) are analysed by Western blot for the presence of ProDer p 3

FIG. 9

Detection of ProDer p 3 in SD S-PAGE after staining with Coomassie blue.

Track 1: cytoplasmic fraction

Track 2: washing of the insoluble fraction

Track 3: insoluble fraction.

The arrow indicates the position of ProDer p 3.

FIG. 10

Purification of ProDer p 3 by Ni2+-NTA chromatograpy

Bound proteins were eluted by addition of increasing concentrations of imidazol in the starting buffer. Fractions were analyzed by SDS PAGE after coomassie blue staining (left panel) and western blot using an anti-His antibody (right panel).

FT: flow-through, 20, 40, 45, 60, 100: imidazol concentration (mM), R: resin Ni2+-NTA after purification

FIG. 11

Correlation between the IgE reactivity of recombinant ProDer p 3 and natural Der p 3. Immunoplates were coated with 500 ng/well of purified natural Der p 3 or recombinant ProDer p 3 and incubated with sera (diluted 1:8) radioallergosorbent positive to D. pteronyssinus. Bound IgE were detected after incubation with a mouse biotinylated anti-human IgB and alkaline phosphatase-labelled streptavidin, followed by an enzymatic assay. Results are expressed as OD₄₁₀ₙₘ values.

The examples which follow are illustrative but not limiting of the invention. Restriction enzymes and other reagents were used substantially in accordance with the vendors' instructions.

### EXAMPLE I

General Procedures

1.—SDS PAGE and Western Blot Analysis

Proteins were analyzed by SDS-PAGE on 12.5% polyacrylamide gels. After electrophoresis, proteins were transferred onto nitrocellulose membranes using a semi-dry transblot system (Bio-Rad). Membranes were saturated for 30 min with 0.5% Instagel (PB Gelatins) in TBS-T (50 mM Tris HCl pH 7.5, 150 mM NaCl, 0.1% Tween 80) and incubated with mouse polyclonal serum raised against denatured or native ProDer p 1 diluted in blocking solution (1:5000). Immunoreactive materials were detected using alkaline phosphatase-conjugated goat anti-mouse antibodies (Promega, 1:7500) and 5-bromo,4-chloro,3-indolylphosphate (BCIP, Boehringer)/nitroblue tetrazolium (NBT, Sigma) as substrates.

2.—Glycan Analysis

Carbohydrate analysis was carried out with the Glycan Differenciation Kit (Boehringer) using the following lectins: Galanthus nivalis agglutinin (GNA), Sambucus nigra agglutinin (SNA), Maackia amurensis agglutinin (MAA), Peanut agglutinin (PNA) and Datura stramonium agglutinin (DSA). Briefly, purified proteins were transferred from SDS-PAGE onto nitrocellulose membranes. Membranes were incubated with the different lectins conjugated to digoxigenin. Complexes were detected with anti-digoxigenin antibodies conjugated to alkaline phosphatase.

3.—Enzymatic Assays

Enzymatic assays were performed in 50 mM Tris-HCl pH 7, containing 1 mM EDTA and 20 mM L-cysteine at 25° C. in a total volume of 1 ml. Hydrolysis of Cbz-Phe-Arg-7-amino-4-methylcoumarin (Cbz-Phe-Arg-AMC) and Boc-Gln-Ala-Arg-7-amino-4-methylcoumarin (Boc-Gln-Ala-Arg-AMC) (Sigma) (both substrates at a final concentration of 100 μM) was monitored using a SLM 8000 spectrofluorimeter with λₑₓ=380 nm and λₑₘ=460 nm. Assays were started by addition of cysteine activated allergen to a final concentration of 100 nM. Before any assay, purified Der p 1 or ProDer p 1 was incubated with a mixture of aprotinin- and p-aminobenzamidine-agarose resins (Sigma) to remove any putative trace of serine protease activity.

4.—Protein Determination

Total protein concentration was determined by the bicinchoninic acid procedure (MicroBCA, Pierce) with bovine serum albumin as standard.

5.—Der p 1 ELISA

Der p 1 or recProDer p 1 was detected with an ELISA kit using Der p 1 specific monoclonal antibodies 5H8 and 4C1 (Indoor Biotechnologies). The Der p 1 standard (UVA 93/03) used in the assay was at a concentration of 2.5 μg/ml.

6.—IgE-Binding Activity

Immunoplates were coated overnight with Der p 1 or ProDer p 1 (500 ng/well) at 4° C. Plates were then washed 5 times with 100 μl per well of TBS-Tween buffer (50 mM Tris-HCl pH 7.5, 150 mM NaCl, 0.1% Tween 80) and saturated for 1 hr at 37° C. with 150 μl of the same buffer supplemented with 1% BSA. Sera from allergic patients to D. pteronyssinus and diluted at ⅛ were then incubated for 1 hr at 37° C. Out of the 95 sera used in the experiments, 16 sera ranged in their specific anti-D. pteronyssinus IgE values (RAST assays) from 58.1 kU/L to 99 kU/L and 79 above the upper cut-off value of 100 kU/L. Plates were washed 5 times with TBS-Tween buffer and the allergen-IgE complexes were detected after incubation with a mouse anti-human IgE antibody (Southern Biotechnology Associates) and a goat anti-mouse IgG antibody coupled to alkaline phosphatase (dilution 1/7500 in TBS-Tween buffer, Promega). The enzymatic activity was measured using the p-nitrophenylphosphate substrate (Sigma) dissolved in diethanolamine buffer (pH 9.8). OD₄₁₀ₙₘ was measured in a Biorad Novapath ELISA reader.

For IgE inhibition assays, plates were coated with Der p 1 or ProDer p 1 at the same concentration (0.12 μM). A pool of 20 human sera from allergic patients (RAST value>100 kU/L) was preincubated overnight at 4° C. with various concentrations (3.6-0.002 μM) of Der p 1 or recProDer p 1 as inhibitors and added on ELISA plates. IgE-binding was detected as described above.

7.—Histamine Release

The histamine release was assayed using leukocytes from the peripheral heparinized blood of an allergic donor and by the Histamine-ELISA kit (Immunotech). Basophils were incubated with serial dilutions of recProDer p 1 or Der p 1 for 30 min at 37° C. The total amount of histamine in basophils was quantified after cell disruption with the detergent IGEPAL CA-630 (Sigma).

8.—ProDer p 1 Denaturation

Recombinant ProDer p 1 was heat-denatured for 5 min at 100° C. in presence of 50 mM β-mercaptoethanol.

9.—Immunisations

Groups of ten CBA/J mice (six weeks old) were four weekly immunised with 5 μg of different proteins or 100 μg of different plasmidic DNA. The purified allergens were injected in presence of alum as adjuvant. As controls, groups of mice were immunised with alum or pJW4304 DNA vector. Mice were bled from the retro-orbital venous plexus on days 7, 14, 21, 28 and sera were collected.

10.—Bronchoprovocation

Within 72 h after immunisations, all mice were placed in a Plexiglas chamber (13×19×37.5 cm) and exposed to aerosolised crude D.pteronyssinus extract over a 20-min period for 7 consecutive days. The concentration of crude mite extract was 300 μg/ml. The aerosols were generated by an ultrasonic nebulizer (Syst' AM). The output of the nebulizer was 0.5 ml/min and the mean particle size of the aerosol was between 1 and 5 μm. As control, mice were nebulized with PBS.

11.—Measurement of Der p 1-Specific IgG, IgG1 and IgG2a

Sera were assayed for anti-Der p 1 IgG, IgG1 and IgG2a antibodies by ELISA. Immunoplates were coated with ProDer p 1 (500 ng/well), for 16 hrs at 4° C. Plates were washed 5 times with TBS-Tween (50 mM Tris-HCl pH 7.5, 150 mM NaCl, 0.1% Tween 80) and saturated for 1 hr at 37° C. with 150 μl of the same buffer supplemented with 1% BSA. Serial dilutions of sera in saturation buffer were incubated for 1 hr at 37° C. Plates were washed 5 times with TBS-Tween buffer and antigen-bound antibodies were detected with the second antibody (goat anti-mouse IgG, Promega, USA) coupled to alkaline phosphatase (dilution 1/7500 in TBS-Tween buffer). The enzymatic activity was measured using the p-nitrophenylphosphate substrate (Sigma) dissolved in diethanolamine buffer (pH 9.8). OD₄₁₅ₙₘ was measured in a Biorad Novapath ELISA reader.

Mouse antibody subclass was determined using immunoplates coated as described above and IgG1- or IgG2a-specific biotin-labelled monoclonal antibodies (rat anti-mouse, dilution 1/7000 in TBS-Tween buffer and 1% BSA, Biosource) as second antibodies. Phosphatase alkaline-conjugated streptavidin ( 1/1000 dilution, Amersham) was added to each well. Assay of the enzymatic activity proceeded as described above.

In all cases, ELISA titers were identified as the reciprocal of the dilution giving a signal corresponding to 50% of the maximal O.D.₄₁₅ value.

12.—Measurement of Der p 1-Specific IgE

Immunoplates were coated with rat anti-mouse IgE (10 ng/well), for 16 hrs at 4° C. Plates were washed 5 times with TBS-Tween (50 mM Tris-HCl pH 7.5, 150 mM NaCl, 0.1% Tween 80) and saturated for 1 hr at 37° C. with 150 μl of the same buffer supplemented with 1% BSA. Serial dilutions of sera in saturation buffer were incubated for 1 hr at 37° C. ProDer p 1 was then added at 500 ng/ml in saturation buffer. Bound ProDer p 1 was detected by addition of biotinylated anti-Der p 1 monoclonal antibody 4C1 (Indoor Biotechnologies) Plates were washed 5 times with TBS-Tween buffer and antibodies-bound antigen were detected with addition of streptavidin coupled to alkaline phosphatase (dilution 1/7500 in TBS-Tween buffer). The enzymatic activity was measured using the p-nitrophenylphosphate substrate (Sigma) dissolved in diethanolamine buffer (pH 9.8). OD₄₁₅ₙₘ was measured in a Biorad Novapath ELISA reader.

13.—Proliferation Assays

To measure Der p 1-specific T-cell proliferative response, immunised mice were sacrificed before and after bronchoprovocations. Lymphocytes were isolated from spleens. Cells (4×10⁵/well in triplicate), cultured in RPMI 1640 with 10% FCS containing 15 mM HEPES and 30 μM β-mercaptoethanol, were stimulated with serial dilutions of crude mite extract or ProDer p 1 in 96-well plates (10 base 2 dilutions of the antigen were tested, starting from a concentration of 25 μg/ml). As control, cells were incubated with only RPMI medium. After 4 days, cells were pulsed with 1 μCi/well [³H] thymidine (Amersham) for 16 hours. Cells were harvested and ³H-thymidine uptake was measured by scintillation counting. Proliferative responses were calculated as the means of quadruplicate wells and were expressed as stimulation index (SI). A stimulation index of >2 was considered positive.

14.—Cytokines Assay

The level of IFNγ and IL-5 in the lymphocyte culture supernatants were measured in ELISA assays. Plates were coated with 1 μg/ml of anti-mouse IL-5 monoclonal (PharMigen) or anti-mouse IFNγ (Biosource) polyclonal antibodies. Plates were washed 5 times with TBS-Tween and saturated for 1 hr at 37° C. with 150 μl of TBS-Tween-BSA. Serial dilutions of splenocyte culture supernatants were added and incubated for 90 min at 37° C. Biotinylated anti-mouse IL-5 (PharMingen, 1 μg/ml) or anti-mouse IFNγ (Biosource, 0.2 μg/ml) antibodies were applied to the plates for 1 h at 37° C. The antigen-antibody complexes were detected by incubation with streptavidin coupled to horseradish peroxydase (dilution 1/10000, Amersham). The enzymatic activity was measured using tetramethylbenzidine (TMB) as substrate (Sigma). The absorbance at 460 nm was measured in a Biorad Novapath ELISA reader. Cytokine concentrations were determined by interpolation from a standard curve performed with purified mouse IL-5 or IFNγ.

15.—Bronchoalveolar Lavage

Three days after the final aerosol exposure, mice were bled and sacrificed. The lungs were immediately washed via the trachea cannula with 1 ml Hank's balanced salt solution (HBSS) which was instilled and gently recovered by aspiration three times. The lavage fluid was centrifuged at 400 g for 10 min at 4° C. The cell pellet was resuspended in 300 μl Hank's balanced salt solution (HBSS) and cells were counted in a Thoma hemocytometer. Cytospin preparations from 50 μl-aliquots were stained with May-Grünwald Giemsa's stain for differential cell counts.

### EXAMPLE II

Expression of MBP-ProDer p 1 in E. coli

1.—Construction of MBP-ProDer p 1 Expression Vector

The complete synthetic cDNA encoding ProDer p 1 (1-302 aa) (SEQ ID NO:1) was isolated from the eukaryotic expression plasmid pNIV 4846 (a pEE 14-derived expression plasmid carrying humanized ProDer p 1 coding cassette, (M. Massaer et al., International Archives of Allergy and Immunology, 2001, 125:32-43) after digestions with Eag I and Xba I. DNA was blunted using large fragment DNA polymerase (Klenow) before Xba I restriction. The 921 bp fragment was inserted at the Asp 718 (blunted end)-Xba I site of pMAL-c2E (New England Biolabs) to give pNIV4854, downstream of the MBP gene. The amino acid sequence of ProDer p 1, encoded by the cDNA of SEQ ID NO:1, is represented in FIG. 2 (SEQ ID NO:2).

2.—Site-Directed Mutagenesis

Mutagenesis of Der p 1 cysteine residues at position 4, 31 or 65 (mature ProDer p 1 numbering, corresponds to positions 84, 111 or 145 in ProDer p 1) was performed in the plasmid pNIV4854, after the substitution of DNA fragments carrying one of the three cysteine codons by synthetic oligonucleotides containing the mutations. The following oligonucleotides were used:

5′TTAAGACCCAGTTTGATCTCAACGCGGAGACCAACGCCCGTATCAACGGCA ATGCCCCCGCTGAGATTGATCTGCGCCAGATGAGGACCGTGACTCCCATCCG CATGC3′ (forward) and 5′CGGATGGGAGTCACGGTCCTCATCTG GCGCAGATCAATCTCAGCGGGGGCATTGCCGTTGATACTACGGGCGTTGGTC TCCGCGTTGAGATCGAAACTGGGTC3′ (reverse) to generate a 110 bp Afl ll-Sph I fragment for the mutation of cysteine residue 4 to arginine (C4R), 5′CAAGGCGGCCGTGGGTCTTGTTGGGCCTTTTCAGGCGTGGCCGCGACAG AGTCGGCATACCTCGCGTATCGGAATCAGAGCCTGGACCTCGC3′ (forward) and 5′TCAGCGAGGTCCAGG CTCTGATTCCGATACGCGAGGTATGCCGACT CTGTCGCGGCCACGCCTGAAAAGGCCCAACAAGACCCACGGCCGCCTTGCAT G3′ (reverse) to generate a 98 bp Sph I-Blp I fragment for the mutation of cysteine residue 31 to arginine (C31R), 5′TGAGCAGGAGCTCGTTGACCGTGCCTCC CAACACGGATGTCATGGGGATACGATTCCCAGAGGTATCGAATACATCCAGC ATA3′ (forward) and 5′CTGGATGTATTCGATACCTCTGGGAATCGTAT CC CCCATGACATCCGTGTTGGGAGGCACGGTCAACGCGCTCCTGC3′ (reverse) to generate a 82 bp Afl II-Sph I fragment for the mutation of cysteine residue 65 to arginine (C65R).

The resulting plasmids containing the ProDer p 1 cassette downstream to the MBP gene and carrying respectively the mutations C4R, C31R and C65R were called pNIV4870, pNIV4871 and pNIV4872. All the three mutations were verified by DNA sequencing. Mutated ProDer p 1 amino acid sequences respectively carrying C4R, C31R and C65R mutation are illustrated in SEQ ID NO:3, SEQ ID NO:5 and SEQ ID NO:7 respectively. The corresponding encoding nucleic acid sequences are shown in SEQ ID NO:4 (C4R mutation), SEQ ID NO:6 (C31R mutation) and SEQ ID NO:8 (C65R mutation).

3.—Expression and Purification of Wild-Type and Mutant MBP-ProDer p 1

E. coli containing the different recombinant expression vectors were grown overnight at 37° C. in 869 medium (A. Jacquet et al., Prot. Exp. Purif. 1999, 17, 392-400) with 100 jig/ml ampicillin. Cells were then diluted 1:100 and allowed to grow at 37° C. to an optical density between 0.4 and 0.6 at 600 nm. Isopropyl β-D-thiogalactoside (IPTG) was added to a final concentration of 0.3 mM. After a 2 h period of induction, cells were harvested by centrifugation at 10000 rpm for 15 min.

Bacterial cell pellets from 1 liter cultures were resuspended in 20 mM Tris-HCl pH 7.5, containing 1 mM aprotinin and AEBSF, and broken under a pressure of 1800 bars using a Cell disrupter (Constant Systems Ltd, Warwick, UK). The lysate was ultracentrifugated at 150,000 g for 60 min. The pellet resulting from the ultracentrifugation was washed with 20 mM Tris-HCl pH 7.5. Insoluble proteins were extracted overnight at 4° C. with 20 mM Tris-HCl pH 7.5 containing 6M urea. The suspension was ultracentrifugated at 150,000 g for 60 min. The supernatant was directly dialysed overnight against 20 mM Tris-HCl pH 7.5, 200 mM NaCl, 1 mM EDTA. The solution was centrifugated to remove any precipitated protein and directly applied onto an amylose resin (1×15 cm) equilibrated in the same buffer. The column was washed with the starting buffer until the A₂₈₀ₙₘ reached the baseline. Proteins were eluted by the addition of 10 mM maltose in the column buffer. Fractions containing the fusion proteins were pooled and concentrated. Purified proteins were stored at −20° C.

### EXAMPLE III

Expression of Three Different ProDer u 1 Mutants in CHO Cells

1.—Site-Directed Mutagenesis

Mutations of Der p 1 cysteine residues at position 4, 31 or 65 (mature Der p 1 numbering, corresponds to positions 84, 111 or 145 in ProDer p 1) were introduced into the plasmid pNIV4846. Plasmids pNIV4870, pNIV4871 and pNIV4872, containing the Der p 1 cassette downstream to the MBP (see Example II) gene and carrying respectively the mutations C4R, C31R and C65R were each restricted with SfuI-XhoI to isolate a 714 bp fragment. The purified DNA fragments were inserted into plasmid p4846 previously cleaved with the same restriction enzymes. The resulting plasmids containing the Der p 1 variants C4R, C31R and C65R were called pNIV4873, pNIV4875 and pNIV4874.

2.—Transient Transfections and Selection of ProDer p 1-Producing Stable CHO-K1 Lines.

To determine the production of Der p 1 by plasmids pNIV4873, pNIV4875 and pNIV4874, COS cells were transiently transfected by lipofection. For stable Der p 1 expression, CHO-K1 cells were transfected with the different plasmids by lipofection. After a 3-weeks 25 μM methionylsulphoximin (MSX) selection, one round of gene amplification was carried out with 100 μM MSX.

### EXAMPLE IV

Denatured ProDer p 1 Displays IgG but not IgE-Binding Reactivity Towards Allergic Sera.

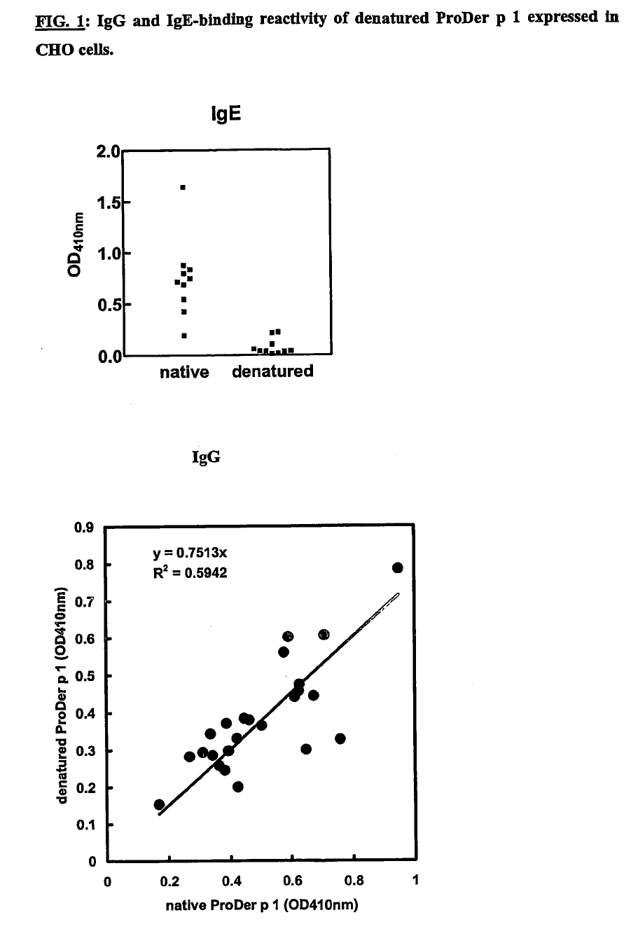

To determine whether a denatured form of ProDer p 1 could be used as a hypoallergenic vaccine, IgG- and IgE binding reactivities of denatured (5 min at 100° C. in the presence of 50 mM P-mercaptoethanol) ProDer p 1 were assayed in ELISA tests. As shown in FIG. 1, denatured ProDer p 1 conserved the main part of the IgG epitopes present on native ProDer p 1. On the other hand, the denatured allergen highly lost its IgE-binding reactivity. Our data suggest that denatured ProDer p 1 could represent a hypoallergenic variant of ProDer p 1.

### EXAMPLE V

IPE Reactivities of MAP-ProDer p 1.

The aim of the experiment was to compare the IgE reactivity of MBP-ProDer p 1 and of natural Der p 1. The reactivity of MBP-ProDer p 1 with specific IgE from sera of allergic patients was assessed in a direct ELISA wherein immunoplates were directly coated with Der p 1 or MBP-ProDer p 1. FIG. 2 shows a strong correlation between the IgE binding to Der p 1 and MBP-ProDer p 1.

### EXAMPLE VI

IgE-Binding reactivities of MBP-ProDer p 1 Mutants.

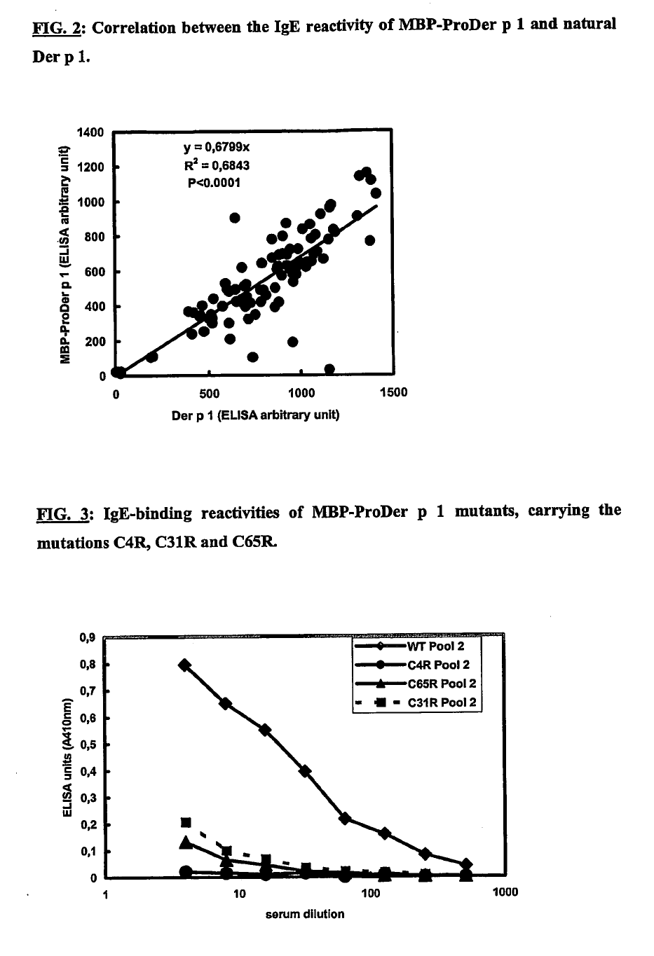

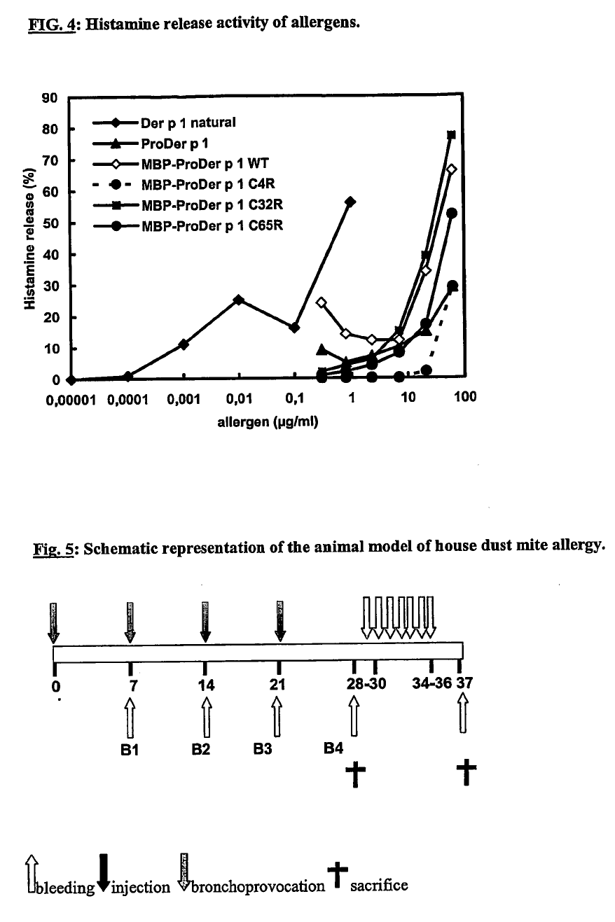

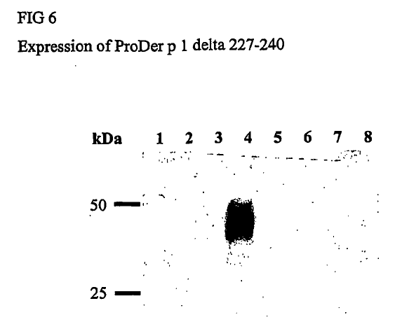

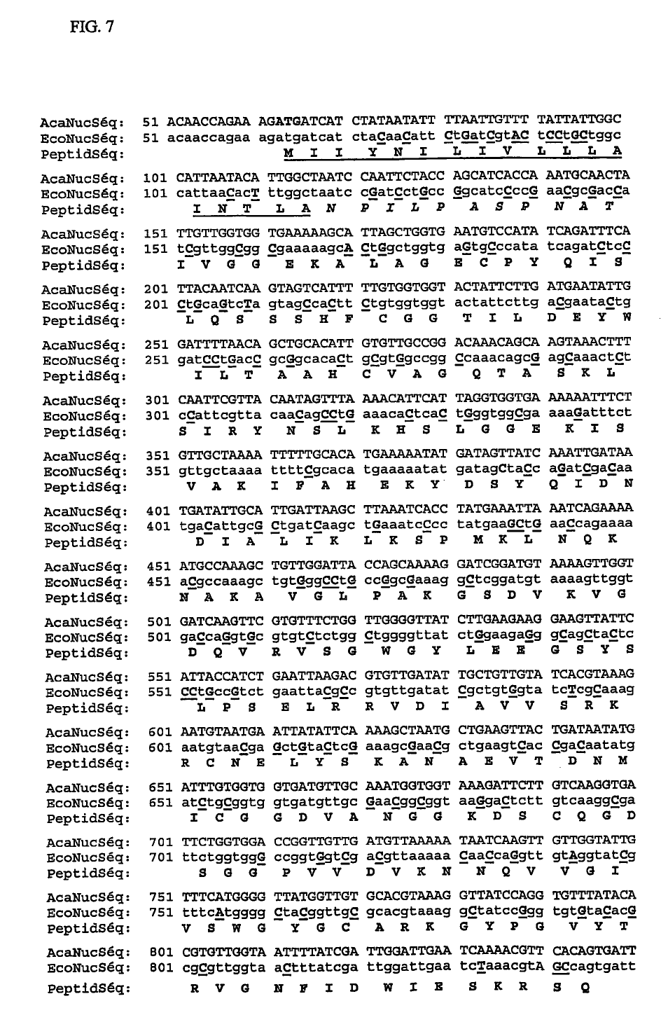

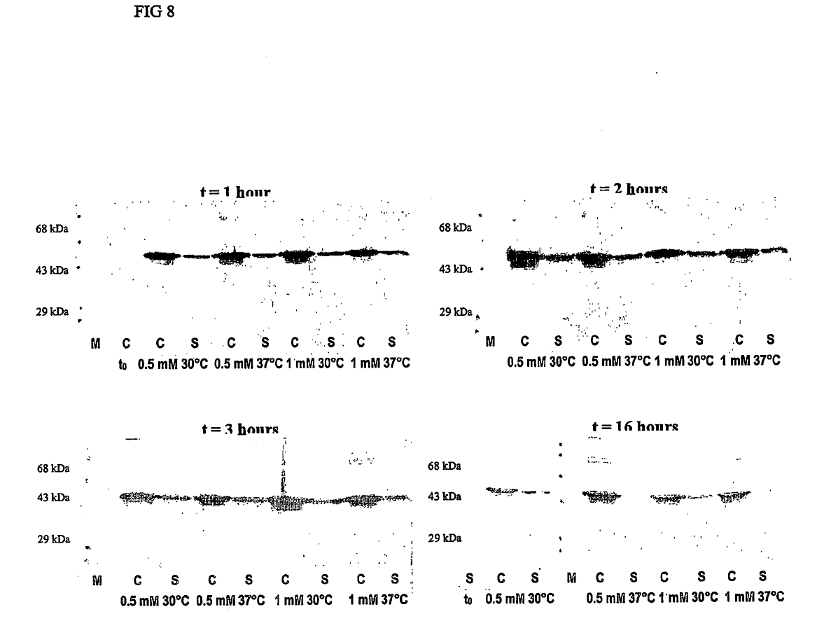

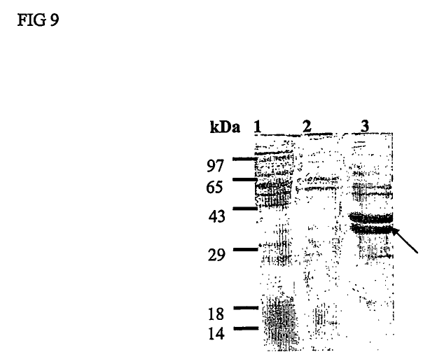

The IgE-binding capacity of MBP-ProDer p 1 mutants was determined in direct ELISA assays for which immunoplates were directly coated with the different forms of MBP-ProDer p 1. A serum pool, made from 20 individual D. pteronyssinus-allergic patient sera with RAST value >100 kU/L, were used in the assays. As shown in FIG. 3, the IgE binding reactivity of the variants C31R and C65R drastically decreased to 5% compared with that of wild-type MBP-ProDer p 1. Strikingly, no reactivity (0% left) of IgE to MBP-ProDer p 1 was observed when residue cysteine 4 was mutated to arginine. The IgE reactivities were specific of the ProDer p 1 moiety as there were no IgE-mediated immune recognitions of MBP or MBP in fusion with an irrelevant protein. Similar results were obtained with another serum pool from 20 others patients.

### EXAMPLE VII

Histamine Release Activity of Various Forms of ProDer p 1.

To compare the allergenic activity of natural Der p 1 with that of recombinant mutated derivatives of ProDer p 1, basophils from one allergic patient were challenged in vitro with various concentrations of allergens and the released histamine was measured. As shown in FIG. 4, natural Der p 1 was able to induce histamine release from basophils even at a concentration of 1 ng/ml. By contrast, recombinant mutated forms of ProDer p 1 could only release histamine at a 1000-10000-fold higher concentration, These results clearly showed that ProDer p 1 mutants display lower IgE binding reactivity than does the natural Der p 1.

### EXAMPLE VIII

Immunogenicity Experiments with Various Forms of ProDer p 1.

1.—Animal Model of House Dust Mite Allergy

An animal model of house dust mite allergy has been developed. CBA/J mice were injected with purified Der p 1 adjuvanted with alum. After four injections at one week interval, animals were subjected to a series of bronchoprovocation with D. pteronyssinus extract (FIG. 5). This model was used to test different recombinant forms of Der p 1 as well as different DNA as prophylactic vaccines against house dust mite allergy.

### EXAMPLE IX

Expression of a Deleted Form of ProDer p 1.

1) The deletion was done by PCR and using synthetic oligonucleotides comprising sequences downstream and upstream of the fragment to be deleted. A Snab I/Avr II fragment of the plasmid pNIV4878 (remember, pNIV4878 is a pPIC9K plasmid (Invitrogen) containing the cDNA of ProDer p 1 (humanised cDNA) and favourable for expression of the allergen in P. pastoris) was replaced by the amplified and deleted fragment. P. pastoris yeasts were transformed by this recombinant plasmid and after selection with geneticine (G418), clones resistant at 0.25 mg/ml G418 were isolated. Given that the ProDer p 1 cassette is situated downstream of a signal sequence, we tested the expression of ProDer p 1 A227-240 secreted after induction with methanol for 24 or 48 hours.

2) Introduction of the deletion by PCR

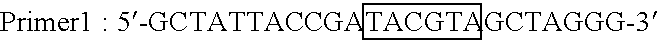

This primer comprises the SnabI restriction site downstream of the zone to be deleted.

Primer2:
5′-CCGTTGTCGCGATCCTTGATTCCGATGATGACAGCG-3′

This primer is therefore homologous to part of the ProDer p 1 sequence, that downstream and upstream of the zone to be deleted.

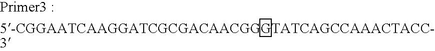

This primer is also homologous to part of the ProDer p 1 sequence and will also allow deletion of 42 pb. In addition, it contains a point mutation which will make it possible to modify the EcoRV site.

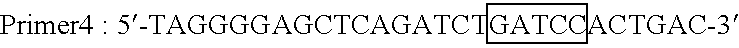

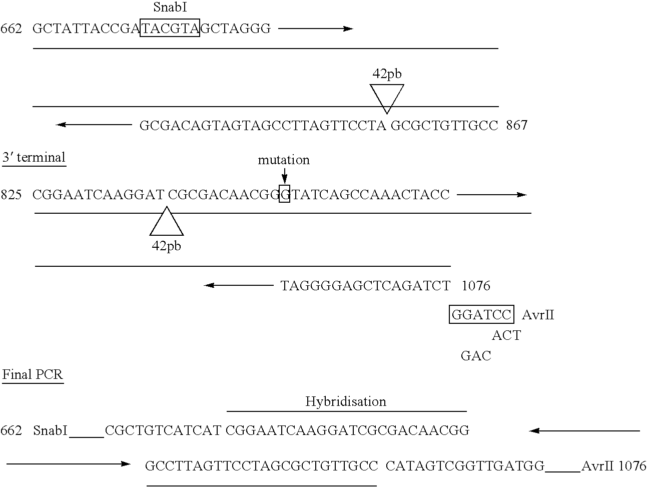

3. Deletion Verified by Sequencing.

The Pichia pastoris yeasts were transformed by the recombinant vector previously linearised by BglII, using the spheroplast method. Transformants were selected for histidinol deshydrogenase (His+) prototrophy. The screening of His+ transformants for geneticin (G418) resistance was performed by plating clones on agar containing increasing concentrations of G418.

(0.25-1-2 and 4 mg/ml). After incubation at 30° C. for several days, we obtained several resistant strains but for only one concentration of 0.25 mg/ml G418.

The expression is induced by adding 0.5% methanol to the culture medium every day. Every day, one millilitre of culture medium is taken in order to recover the supernatant. Expression of ProDer p 1 is displayed by blot.

### EXAMPLE X

Expression of a Triple Mutant Dorm of ProDer p 1.

1) In this example, the cysteine 71, 103 and 117 residues of Der p 1 are mutated into alanine. The Cys→Ala mutation breaks the disulphide bridge but does not introduce any positive charge into the structure of Der p 1. This gentler destructuring might not affect the expression and secretion of ProDer p 1. These three mutations are introduced by PCR, thanks to an oligonucleotide comprising the three mutations.

### EXAMPLE XI

Construction of a PreProDer p 3 Allergen

1. Construction of a PreProDer p 3 Synthetic cDNA

A PreProDer p 3 cDNA was synthesised using a set of 10 partially overlapping oligonucleotides. These primers were designed, based on the codon preference of highly expressed E. Coli bacterial genes, and produced by a 394 DNA/RNA Applied Biosystem synthetizer. The degenerately encoded amino acids were not encoded by the most prevalent codons but taking the frequencies of the individual codons into account. For example, AAG or AAA encodes the lysine residue with a respective frequency of 21.45% and 78.55% in highly expressed E. Coli bacterial genes. Consequently, we attempted to follow the same codon frequency instead of selecting only the AAA codon for each lysine residue in the synthetic PreProDer p 3. The oligonucleotides were the following:

5′ TCATGATCATCTACAACATTCTGATCGTACTCCTGCTGGCCATTAAC
ACTTTGGCTAATCCGATCCTGCCGGCATCCCCGAACGCGACCATCGTTGG
C 3′ (oligo 1, coding)
5′ CACCACAGAAGTGGCTACTAGACTGCAGGGAGATCTGATATGGGCAC
TCACCAGCCAGTGCTTTTTCGCCGCCAACGATGGTCGCG 3′
(oligo 2, noncoding)
5′ GTAGCCACTTCTGTGGTGGTACTATTCTTGACGAATACTGGATCCTG
ACCGCGGCACACTGCGTGGCCGGCCAAACAGCGAGCAAACTCTCC 3′
(oligo 3, coding)
5′ GTCGATCTGGTAGCTATCATATTTTTCATGTGCGAAAATTTTAGCAA
CAGAGATCTTTTCGCCACCCAGTGAGTGTTTCAGGCTGTTGTAACGAATG
GAGAGTTTGCTCGCTG 3′ (oligo 4, noncoding)
5′ GATAGCTACCAGATCGACAATGACATTGCGCTGATCAAGCTGAAATC
CCCTATGAAGCTGAACCAGAAAAACGCCAAAGCTGTGGGCCTGC 3′
(oligo 5, coding)
5′ CAGACGGCAGGGAGTAGCTGCCCTCTTCCAGATAACCCCAGCCAGAG
ACACGCACCTGGTCACCAACTTTTACATCCGAGCCTTTCGCCGGCAGGCC
CACAGCTTTG 3′ (oligo 6, noncoding)
5′ CTACTCCCTGCCGTCTGAATTACGCCGTGTTGATATCGCTGTGGTAT
CTCGCAAAGAATGTAACGAGCTGTACTCGAAAGCGAACGCTGAAGTCAC
3′ (oligo 7, coding)
5′ CCACCAGAATCGCCTTGACAAGAGTCCTTACCGCCGTTCGCAACATC
ACCACCGCAGATCATATTGTCGGTGACTTCAGCGTTCGC 3′
(oligo 8, noncoding)
5′ CAAGGCGATTCTGGTGGGCCGGTGGTCGACGTTAAAAACAACCAGGT
TGTAGGTATCGTTTCCTGGGGCTACGGTTGCGCACGTAAAGGC 3′
(oligo 9, coding)
5′ AAGCTTTCAGTGGTGGTGGTGGTGGTGCTGGCTACGTTTAGATTCAA
TCCAATCGATAAAGTTACCAACGCGCGTGTACACACCCGGATAGCCTTTA
CGTGCGCAAC 3′ (oligo 10, noncoding).

The oligonucleotides were incubated together for the amplification of a synthetic recPreProDer p 3 cDNA in a PCR reaction. PCR was conducted using Expand High Fidelity PCR System (Roche Diagnostics) with the following conditions: 30 cycles, denaturation at 94° C. for 30 s, annealing at 52° C. for 30 s and elongation at 72° C. for 30 s. The generated products were amplified using the 3′ and 5′ terminal primers (oligo 1 and 10) in the same conditions. The resulting 812 bp fragment was cloned into a pCRII-TOPO cloning vector (Invitrogen).

Digestion with BamHI showed that clones 1, 3 and 9 were correctly inserted.

Intra recPreProDer p 3 oligonucleotides were used to sequence the insert:

5′ AAGCTGAAATCCCCTATGAAGC3′ (coding)
5′ CTCTTCCAGATAACCCCAGCC3′ (noncoding).

Only clone 3 proved to be correct, but missing the first 6 bases on 5′ coding end. The addition of the missing bases was achieved by the use of two new oligonucleotides:

5′TTTTATTCATGATCATCTACAACATTCTGATCC3′
(oligo 11, coding)
5′GATGCATGCTCGAGCGGC3′
(oligo 12, noncoding).

The oligonucleotides were incubated with clone 3 DNA carrying the incomplete PreProDer p 3 sequence. The amplification of the synthetic gene was obtained by a PCR reaction using Expand High Fidelity PCR System (25 cycles: denaturation at 94° C. for 30 s, annealing at 53° C. for 30 s and elongation at 72° C. for 30 s). Resulting fragment was cloned into a pCRII-TOPO. The PreProDer p 3 cDNA was isolated after the double Rca I-XhoI restriction and cloned into pET15b expression vector digested with NcoI and XhoI. Competent AD494(DE3)pLys E Coli cells were transformed by the resulting plasmid and 1 mM final concentration isopropyl-thiogalactoside (IPTG) (Duchefa) was added to the culture medium to detect ProDer p 3 expression.

The deletion of the putative Der p 3 signal peptide was performed by PCR and using two new primers: 5′CATATGAATCCGATCCTGCCGGCATCCCC3′ (oligo 13, coding) and 5′GGATCCTCACTGGCTACGTTTAGATTCAATCC3′ (oligo 14, non coding) Amplification of the ProDer p 3 cDNA was done by PCR with Taq Polymerase (Roche Diagnostics), 15 cycles: denaturation at 97° C. for 30 s, annealing at 65° C. for 30 s and elongation at 72° C. for 1 min. The resulting 750 bp fragment was cloned into a pCRII-TOPO cloning vector (Invitrogen). Top 10 competent E Coli were transformed by the resulting plasmid. 9 clones appeared positively inserted; digestion with EcoRi proved clones 1,4,8 to be correctly inserted, while sequencing showed that only clone 4 had the right sequence. The ProDer p 3 cDNA was isolated after the digestions with NdeI and XhoI and cloned into pET15b digested by the same enzymes. The BL21 and BL21 Star E Coli (Invitrogen) strains were transformed by the resulting plasmid Addition of IPTG in the culture medium induced the expression of recombinant ProDer p 3 carrying (HiS)₆ tag at its N-terminal end.

2. Expression of the Recombinant Allergen in E Coli

The best producing recombinant E. Coli BL21 STAR clone was cultured (37°/250 rpm) in 2 liters liquid 869 medium containing 100 μg/ml ampicillin (Pentrexyl). When the culture absorbance at 620 nm reached 0.5, expression was induced for three hours by the addition of 1 mM final concentration isopropyl-thiogalactoside (Duchefa). The culture was then harvested and centrifuged at 11000 g and stored at −20° C. Bacterial pellet was recovered and resuspended in 40 ml Tris buffer 20 mM pH 7.5, implemented with Aprotinin 1/1000 (Sigma) and AEBSF 1/500 (ICN). Followed the crush of bacteria at 1500 bars and the storage at −20° C.

3. Purification of recProDer p 3 from Crushed E. Coli

Harvested E. coli cells, resuspended in 20 mM Tris pH 7.5, aprotinin 1 mM and AEBSF 1 mM, were lysed through a cell disrupter (Cell D) and under a pressure of 1800 kbars.

The lysate was ultracentrifuged at 149000 g for 1 h. The supernatant was removed and the pellet containing recombinant Proder p 3 was subsequently extracted overnight at 4° C. with 40 ml of 50 mM Tris-HCl buffer containing 6M Guanidine Hydrochloride 6M pH 7.5. After ultracentrifugation (45′, 149000 g), the supernatant of extraction was applied at 3 ml/min on a Nickel-NTA Superflow column (1.6×5 cm, Qiagen) equilibrated with the extraction buffer. The column was washed at 1.5 ml/min with PBS NaCl 0.5M pH 7.5 to renaturate boun proteins. RecProDer p 3 was eluted at 4 ml/min by addition of 200 mM imidazole in the conditioning buffer. Fractions containing recProDer p 3 were pooled, concentrated by ultrafiltration (Amicon-Millipore regenerated cellulose ultrafiltration membranes, NMWL 10 kDa). During this step, the buffer was exchanged by PBS pH 7.3. Purified protein was stored at −20° C.

4. Purification of Natural Der p 3 from Natural Mite Whole Body Extracts

D. pteronyssimus extracts were submitted to a 60% final saturation (NH₄)₂SO₄ precipitation. After ultra centrifugation (45′, 149000 g), the supernatant was applied at 2 ml/min on a Benzamidine Sepharose 4 fast flow column (1.6×5 cm, Pharmacia) equilibrated with Tris buffer 50 mM NaCl 0.5M pH 7.4. Der p 3 was eluted from the column with 50 mM Glycine-HCl buffer pH2.5 and each 1 ml fraction was immediately neutralized by the addition of 75 μl Tris 1M pH 9.5. Fractions containing Der p 3 were pooled, concentrated (Amicon-Millipore regenerated cellulose ultrafiltration membranes, NMWL 10 kDa) and applied at 0.5 ml/min on a Superdex 75 gel filtration chromatography column (Pharmacia) equilibrated with PBS pH 7.3. Purified Der p 3 was concentrated and stored at −20° C.

5. SDS PAGE and Western Blot Analysis

Proteins were analysed by SDS-PAGE on 12.5% polyacrylamide gels. After electrophoresis, proteins were transferred onto nitrocellulose membranes using a semi-dry transblot system (Sigma-Aldrich). Membranes were saturated for 30 min with 0.5% Instagel (PB Gelatins) in TBS-T (50 mM Tris HCl pH 7.5, 150 mM NaCl, 0.1% Tween 80) and incubated with mouse polyclonal serum raised against ProDer p 3 diluted in blocking solution (1:2500). Immunoreactive materials were detected using alkaline phosphatase-conjugated goat anti-mouse antibodies (Promega, 1:7500) and 5-bromo,4-chloro,3-indolylphosphate (BCIP, Boehringer)/nitroblue tetrazolium (NBT, Sigma) as substrates.

6. Protein Determination

Total protein concentration was determined by the bicinchoninic acid procedure (MicroB CA, Pierce) with bovine serum albumin as standard.

7. IRE-Binding Activity.

Immunoplates were coated overnight with Der p 3 or recProDer p 3 (500 ng/well) at 4° C. Plates were then washed 5 times with 100 μl per well of TBS-Tween buffer (50 mM Tris-HCl pH 7.5, 150 mM NaCl, 0.1% Tween 80) and saturated for 1 hr at 37° C. with 150μl of the same buffer supplemented with 1% BSA (Sigma). Sera from allergic patients to D. pteronyssinus and diluted at ⅛ were then incubated for 1 hr at 37° C. Out of the 47 sera used in the experiments, 5 sera ranged in their specific anti-Dpteronyssinus IgE values (RAST assays) from 0.7 kU/L to 28.9 kU/L, 8 from 68.3 kU/L to 94.1 kU/L and 34 above the upper cut-off value of 100 kU/L. Plates were washed 5 times with TBS-Tween buffer and the allergen-IgE complexes were detected after incubation with a biotinconiugate mouse anti-human IgE antibody (dilution 1/2000 in TBS-T buffer, Southern Biotechnology Associates) and streptavidin-horseradish peroxydase (dilution 1/1000 in TBS-Tween buffer, Amersham Life Science). The enzymatic activity was measured using the 3,3′,5,5′-tetramethyl-benzidine (TMB) (Sigma). OD₄₅₀ₙₘ was measured in a Biorad Novapath ELISA reader.

8. Enzymatic Assays

Enzymatic assays were performed in 50 mM Tris-HCl pH 8, at 25° C. in a total volume of 200 μl. Hydrolysis of N-α-benzoyl-L-argine-p-nitroanilide (Sigma) (final concentration 1 mM) was measured by a Biorad Novapath ELISA reader at 405 nm. Assays were started by the addition of the allergen at the final concentration of 6.25 μg/ml.

9. Cloning and Expression of Der p 3

Expression in Escherichia coli.

An immature form of Der p 3, ProDer p 3, was expressed in bacteria. The cDNA of PreProDer p 3 was synthesised completely, with the help of 12 synthetic oligonucleotides and with use of codons optimised for expression in bacteria (FIG. 2). The cassette coding for ProDer p 3 was cloned in the expression vector pET-15b downstream of a sequence coding for a poly-histidine tail. This vector contains a T7 promoter inducible by adding IPTG to the culture medium. Firstly, we tried to optimise the expression conditions of ProDer p 3. To do this, cultures of recombinant bacteria were incubated at 30° C. and 37° C., with two concentrations of inducing agent (IPTG 0.5 and 1 mM) for periods of 1, 2, 3 and 16 hours. Each bacterial pellet was lysed in the French press. The lysates were centrifuged at 20000 rpm for 20 min. The presence of ProDer p 3 in the supernatants (cytoplasm fractions) and/or the pellets (debris and insoluble products) was revealed by Western blot using a mouse antibody against the poly-histidine tail of the recombinant ProDer p 3 (FIG. 3).

The expression test shows only a slight effect of temperature and IPTG concentration on the production of ProDer p 3. ProDer p 3 is essentially expressed in insoluble form, its expression is optimal for an induction period of 2 to 3 h. The absence of a soluble form of ProDer p 3 for a 16-hour induction should be noted. ProDer p 3 is expressed because it is detected after staining with Coomassie blue. In SDS-PAGE, the protein is in the form of a band of ±32 kDa (FIG. 4).

10. Purification of ProDer p 3

A protocol for purification of recombinant ProDer p 3 has been developed.

Given that ProDer p 3 is expressed in insoluble form, the recombinant allergen is solubilised by extraction of the pellet in denaturing conditions (Tris-HCl 20 mM, guanidine chloride 6 M pH 7.5). The extraction yield is in the order of 80-90%. The extract is applied to about 10 ml of a column of Ni²⁺-chelating sepharose resin (1.6 cm diameter, 5 cm high) packed in the extraction buffer. After washing the column with the extraction buffer, the attached proteins are renatured directly on the column by a linear renaturation gradient with the buffer PBS 0.5 M NaCl pH 7.5. The volume of the gradient is 200 ml.

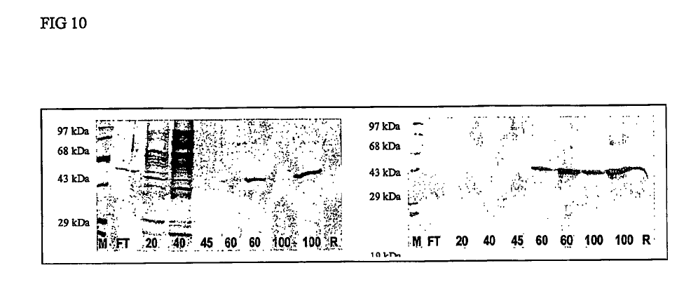

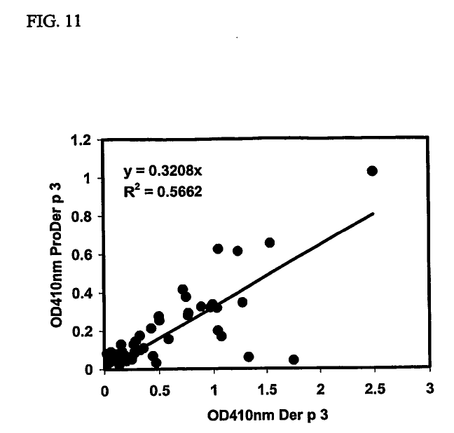

The elution is then carried out by application of increasing concentrations of imidazole in the renaturation buffer (20, 50, 100 et 200 mM). Analysis by SDS-PAGE shows that the protein is not present in the effluate and that the contaminants elute at an imidazole concentration of 20 mM while ProDer p 3 elutes between 60 and 100 mM imidazole. All the ProDer p 3 is detached from the chromatographic support (see FIG. 10).

The analysis of the N-terminal sequence of ProDer p 3 was done by micro-sequencing. The sequence corresponds to that of the histidine tail. It should be noted that in the vector pET-15b, the cloning site of the ProDer p 3 is downstream of the sequence coding for the histidine tail. ProDer p 3 and the histidine tail are separated by a thrombin restriction site. In order to verify the authenticity of ProDer p 3, we treated the purified protein with thrombin in order to eliminate the histidine tail. A second microsequencing of the digested ProDer p 3 revealed the N-terminal sequence of the propeptide of Der p 3 (N P I L P A S P N A T).

Enzymatic activity: ProDer p 3 is inactive against BAPNA, a substrate restrictable by natural Der p 3.

11. IgE Reactivity

Direct coating of allergens.

This preliminary result seems to indicate a lower reactivity of ProDer p 3 compared with Der p 3 in relation to the IgE of patients allergic to mites. (FIG. 11)

2.—Vaccine Formulations

TABLE 1
protein and DNA vaccine formulations tested in the house dust
mite allergy animal model depicted in FIG. 5.

| Protein | DNA | Adjuvant | Way of injection |
|---|---|---|---|
| Natural Der p 1 | | Alum | IP |
| ProDer p 1 native | | Alum | IP |
| ProDer p 1 native | | — | IM |
| ProDer p 1 denatured | | Alum | IP |
| MBP-ProDer p 1 | | Alum | IP |
| MBP-ProDer p 1 C4R | | Alum | IP |
| MBP-ProDer p 1 C31R | | Alum | IP |
| MBP-ProDer p 1 C65R | | Alum | IP |

IP = intraperitoneal injection
IM = intramuscular injection

3.—Antibody Response—Results

Mice immunized by four injections of natural Der p 1 produced high titers of IgG and IgG1, low titers of IgG2a and large amounts of IgE antibodies, indicating that natural Der p 1 induces strong Th2 immunes responses (Tables 2 and 4).

The anti-Der p 1 IgG and IgG1 antibody responses were also strong in mice injected with native or denatured ProDer p 1. After injections with native ProDer p 1, the IgG2a titers were slightly higher than those obtained with Der p 1, IgE titers being comparable or slightly lower than those obtained with Der p 1. In contrast to the native ProDer p 1-immunized mice, animals injected with denatured ProDer p 1 produced high IgG2a titers and very low IgE antibodies. As expected, immunizations with ProDer p 1 in the absence of Alum induced poor immune responses (Table 4).

MBP-ProDer p 1 wild type (WT), C4R, C31R and C65R-sensitized mice showed similar productions of specific IgG and IgG1 antibodies (Table 3). Highest IgG2a titers were observed in groups immunized with MBP-ProDer p 1 WT and C31R.

Specific IgE titers were low, whatever the MBP-ProDer p 1 variants injected.

Similar results were obtained after mice immunizations with plasmid encoding ProDer p 1.

TABLE 2
Titers of specific anti-Der p 1 antibodies from mice immunized
with different antigens. For IgE titers, results are expressed
as OD_{415 nm} values for a 1/10 dilution of sera. Titers
were also measured after bronchoprovocations with PBS
or with D. pteronyssinus extracts (HDM).

| Antigen | Bleeding | Challenge | IgG | IgG1 | IgG2a | IgE |
|---|---|---|---|---|---|---|
| Der p 1 | 1 | | <50 | <50 | <50 | 0 |
| | 2 | | 214 | 900 | <50 | 1.1 |
| | 3 | | 700 | 6062 | <50 | 0.2 |
| | 4 | | 2500 | 24390 | 100 | 0.6 |
| | 5 | PBS | 8670 | 16340 | 300 | 0.7 |
| | | HDM | 8230 | 17440 | 300 | 0.6 |
| ProDer p 1 | 1 | | <50 | <50 | <50 | 0 |
| native | | | | | | |
| | 2 | | 301 | 1146 | <50 | 1.1 |
| | 3 | | 800 | 6860 | 86 | 0.3 |
| | 4 | | 2500 | 28545 | 203 | 0.5 |
| | 5 | PBS | 8266 | 25500 | 600 | 0.3 |
| | | HDM | 11880 | 38310 | 600 | 0.6 |
| denatured | 1 | | <50 | <50 | <50 | 0 |
| | 2 | | 330 | 861 | 120 | 0.2 |
| | 3 | | 966 | 3402 | 210 | 0.07 |
| | 4 | | 3093 | 14830 | 970 | 0.1 |
| | 5 | PBS | 16380 | 54040 | 2700 | 0.1 |
| | | HDM | 14200 | 32140 | 2700 | 0.05 |

TABLE 3
Titers of specific anti-Der p 1 antibodies from mice immunized
with different antigens. For IgE titers, results are expressed
as OD_{415 nm} values for a 1/10 dilution of sera. Titers
were also measured after bronchoprovocations with PBS
or with D. pteronyssinus extracts (HDM).

| Antigen | Bleeding | Challenge | IgG | IgG1 | IgG2a | IgE |
|---|---|---|---|---|---|---|
| MBP-ProDer | 2 | | 637 | 3351 | 144 | 0.046 |
| p 1 WT | 3 | | 4444 | 24720 | 757 | 0.039 |
| | 4 | | 2500 | 24390 | 100 | 0.6 |
| | 5 | PBS | 6151 | 29500 | 2899 | 0.13 |
| | | HDM | 3437 | 22210 | 1496 | 0.27 |
| MBP-ProDer | 2 | | 583 | 2212 | 95 | 0 |
| p 1 C4R | 3 | | 1123 | 6131 | 356 | 0.021 |
| | 4 | | 2500 | 28545 | 203 | 0.5 |
| | 5 | PBS | 2064 | 9077 | 624 | 0.004 |
| | | HDM | 2418 | 14390 | 635 | 0.029 |
| MBP-ProDer | 2 | | 1221 | 4572 | 144 | 0.017 |
| p 1 C31R | | | | | | |
| | 3 | | 6472 | 40405 | 1311 | 0.029 |
| | 4 | | 3093 | 14830 | 970 | 0.1 |
| | 5 | PBS | 2897 | 10880 | 857 | 0.063 |
| | | HDM | 5508 | 24300 | 1959 | 0.074 |
| MBP-ProDer | 2 | | 202 | 887 | <50 | 0.022 |
| p 1 C65R | | | | | | |
| | 3 | | 1252 | 5718 | 363 | 0.066 |
| | 4 | | 3093 | 14830 | 970 | 0.1 |
| | 5 | PBS | 782 | 3958 | 87 | 0.108 |
| | | HDM | 3109 | 16250 | 430 | 0.117 |

TABLE 4
Titers of specific anti-Der p 1 antibodies from mice immunized
with different antigens. For IgE titers, results are expressed
as OD_{415 nm} values for a 1/10 dilution of sera. Titers were
also measured after bronchoprovocations with PBS
or with D. pteronyssinus extracts (HDM).

| Antigen | Bleeding | Challenge | IgG | IgG1 | IgG2a | IgE |
|---|---|---|---|---|---|---|
| Der p 1 | 2 | | 201 | 1135 | <20 | 0.852 |
| | 3 | | 3264 | 18002 | <50 | 0.34 |
| | 4 | | 8271 | 43306 | <50 | 0.59 |
| | 5 | PBS | 10072 | 57670 | <100 | 0.44 |
| | | HDM | 6058 | 72810 | <100 | 0.68 |
| ProDer p 1 | 2 | | 929 | 7422 | 159 | 0.8 |
| Alum | 3 | | 5061 | 27244 | 586 | 0.37 |
| | 4 | | 15110 | 68960 | 1016 | 0.46 |
| | 5 | PBS | 10900 | 57255 | 1190 | 0.421 |
| | | HDM | 16770 | 79460 | 1125 | 0.485 |
| ProDer p 1 | 2 | | 136 | 774 | <20 | 0.58 |
| (no adjuvant) | | | | | | |
| | 3 | | 1389 | 8571 | 104 | 0.13 |
| | 4 | | 4704 | 14126 | 120 | 0.17 |
| | 5 | PBS | 3587 | 16930 | 105 | 0.28 |
| | | HDM | 3880 | 20737 | 100 | 0.25 |

4.—T-Cell Proliferative Response—Results

Before (control) and after aerosol challenge, splenocytes isolated from immunized mice were examined for T-cell proliferative response by stimulation with ProDer p 1 or D. pteronyssinus extract. Results are shown in Table 5 (stimulation index) and in Table 6 (cytokines).

Allergen-specific T cell responses were detected in immunized mice with the different recombinant ProDer p 1 mutants. Strongest responses were observed when splenocytes were restimulated with ProDer p 1. T-cell reactivities appeared to be independent from the challenge.

These results in Table 5 indicated that the different forms of ProDer p 1 shared common T-cell epitopes with natural Der p 1. Moreover, destructuration of ProDer p 1 by thermal denaturation or site-directed mutagenesis did not alter ProDer p 1 T-cell reactivity, confirming that these forms are hypoallergens with very low IgE-binding reactivity able to stimulated T-cell responses.

TABLE 5
Vaccinated mice were challenged or not with PBS or D. pteronyssinus
extracts. Spleen cells were isolated and restimulated in vitro with
purified ProDer p 1 or with D. pteronyssinus extracts. Stimulation
index was measured by [³H]-thymidine incorporation.

| | Concentration | S.I. (stimul. With | | | S.I. (stimul. with | | |
|---|---|---|---|---|---|---|---|
| | of stimulating | ProDer p 1) | | | HDM ext.) | | |
| | antigen | Aerosol | | | aerosol | | |
| Antigen | (μg/ml) | None | PBS | HDM | None | PBS | HDM |
| MBP-ProDer | 50 | 7.3 | 14.97 | 20.8 | — | — | — |
| p 1 WT | | | | | | | |
| MBP-ProDer | 50 | 19.1 | 9.7 | 16.3 | — | — | — |
| p 1 C4R | | | | | | | |
| MBP-ProDer | 50 | 5.4 | 10.0 | 14.7 | — | — | — |
| p 1 C31R | | | | | | | |
| MBP-ProDer | 50 | 6.8 | 8.8 | 13.0 | — | — | — |
| p 1 C65R | | | | | | | |
| Der p 1 | 40 | — | 1.6 | 17.5 | — | 1.6 | 7.5 |
| ProDer p 1 | 40 | — | 30.9 | 11.5 | — | 2.8 | 2.8 |
| ProDer p 1 | 40 | — | 24.0 | 15.9 | — | 1.7 | 1.4 |
| denatured | | | | | | | |
| Alum | 40 | — | 4.2 | 4.6 | — | 2.0 | 1.3 |

—: not available. These results are obtained from different experiments, not from only one. Consequently, cytokine assays can not be compared between all groups.

The presence of cytokines IL-5 and IFNγ in the culture supernatants of restimulated splenocytes was determined in ELISA (Table 6). If we compared the ratio [IFNγ]/[IL-5], we could conclude that vaccinations with natural Der p 1 or ProDer p 1 adjuvanted with alum induced a better production of IL-5 than IFNγ. The different forms of MBP-ProDer p 1 (mutants and wild-type) as well as denatured ProDer p 1 induced comparable levels of both cytokines.

TABLE 6
[IL-5] and [IFNγ] in supernatants from ProDer p
1-restimulated splenocytes. These results are obtained
from different experiments, not from only one. Consequently,
cytokine assays can not be compared between all groups.

| | [IL-5] (pg/ml) | | | [IFNγ] (pg/ml) | | |
|---|---|---|---|---|---|---|
| | Aerosol | | | Aerosol | | |
| Antigen | none | PBS | HDM | None | PBS | HDM |
| MBP-ProDer p 1 | 420 | 165 | 929 | 987 | 1076 | 1282 |
| MBP-ProDer p | 330 | 51 | 308 | 551 | 1366 | 1177 |
| 1C4R | | | | | | |
| MBP-ProDer p | 430 | 202 | 1141 | 1348 | 1281 | 3392 |
| 1C31R | | | | | | |
| MBP-ProDer p | 0 | 0 | 953 | 0 | 0 | 1161 |
| 1C65R | | | | | | |
| Alum | 0 | 0 | 0 | 0 | 0 | 0 |
| Der p 1 | 75 | 45 | 495 | 0 | 0 | 190 |
| ProDer p 1 | 0 | 355 | 400 | 0 | 125 | 210 |
| ProDer p 1 | — | 850 | 736 | — | 822 | 1119 |
| denatured | | | | | | |

5.—Bronchoalveolar Lavage—Results

Sensitisation with natural Der p 1 and subsequent exposure to aerosolised house dust mite extracts induced significantly higher bronchoalveolar cell numbers (Table 7). Seven exposures to aerosolised house dust mite extracts were shown to induce airway eosinophilia in only the animals vaccinated with Der p 1. In this group, airway eosinophilia was not observed when Der p 1-sensitised animals were not nebulized or exposed to aerosolised PBS.

Vaccinations with the different recombinant forms of ProDer p 1 prevented airway eosinophilia, even after exposure to aerosolised HDM extracts.

TABLE 7
Characterization of the bronchoalveolar lavage
fluid of different antigen-immunized mice exposed
to PBS or house dust mite extracts aerosols

| | | | | | | | Total |
|---|---|---|---|---|---|---|---|
| | | | | | | | cells |
| | | Lympho | Eosino | Neutro | Macro | Mono | (10⁵/ |
| Antigen | Aerosol | (%) | (%) | (%) | (%) | (%) | ml) |
| Der p 1 | none | 86 | 4 | 0 | 6 | 3 | 2.2 |
| | HDM | 13 | 68 | 7 | 6 | 6 | 167 |
| | PBS | 90 | 0 | 2 | 4 | 4 | 4.8 |
| ProDer | none | 90 | 0 | 0 | 7 | 3 | 3.2 |
| p 1 | HDM | 69 | 7 | 12 | 3 | 10 | 5.1 |
| | PBS | 76 | 5 | 4 | 7 | 8 | 7.6 |
| ProDer | none | 51 | 5 | 2 | 22 | 20 | 4 |
| p 1 | | | | | | | |
| denatured | | | | | | | |
| | HDM | 52 | 4 | 26 | 10 | 7 | 6.9 |
| | PBS | 67 | 2 | 2 | 20 | 9 | 5.2 |
| Alum | none | 88 | 1 | 4 | 7 | 0 | 3.6 |
| | HDM | 80 | 0 | 4 | 14 | 1 | 1.5 |
| | PBS | 88 | 1 | 5 | 5 | 1 | 1.2 |
| MBP- | none | 85 | 2 | 4 | 7 | 0 | 1.5 |
| ProDer | | | | | | | |
| p 1 | | | | | | | |
| | HDM | 70 | 3 | 14 | 8 | 5 | 2.1 |
| | PBS | 88 | 1 | 6 | 5 | 0 | 0.6 |
| MBP- | none | 90 | 2 | 4 | 4 | 1 | 2.2 |
| ProDer | | | | | | | |
| p 1 | | | | | | | |
| C4R | | | | | | | |
| | HDM | 71 | 2 | 14 | 11 | 1 | 2 |
| | PBS | 80 | 2 | 7 | 10 | 1 | 4.5 |
| MBP- | none | 79 | 1 | 14 | 7 | 0 | 1.3 |
| ProDer | | | | | | | |
| p 1 | | | | | | | |
| C31R | | | | | | | |
| | HDM | 65 | 4 | 27 | 5 | 1 | 2 |
| | PBS | 87 | 2 | 7 | 5 | 1 | 3 |
| MBP- | none | 85 | 0 | 4 | 10 | 1 | 2.4 |
| ProDer | | | | | | | |
| p 1 | | | | | | | |
| C65R | | | | | | | |
| | HDM | 84 | 1 | 7 | 7 | 1 | 2.4 |
| | PBS | 84 | 1 | 4 | 12 | 0 | 1.5 |

### EXAMPLE XII

Expression Plasmid for Nucleic Acid Vaccination (NAVAC)

1.—Construction of ProDer p 1 Encoding Plasmid for Nucleic Acid Vaccination

The ProDer p 1 coding cassette (1-302aa) was excised from plasmid pNIV4846 (see above), restricted with HindIII and BglII, and inserted into plasmid pJW4304 previously cleaved with HindIII and BglII. The resulting plasmid, named pNIV4868, was verified by DNA sequencing.

2.—Site-Directed Mutagenesis

Mutations of ProDer p 1 cysteine residues at position 4, 31 or 65 (mature Der p 1 numbering, corresponds to positions 84, 111 or 145 in ProDer p 1) were introduced into the plasmid pNIV4868. Plasmids pNIV4870, pNIV4871 and pNIV4872, containing the ProDer p 1 cassette downstream to the MBP gene and carrying respectively the mutations C4R, C31R and C65R were each restricted with AflII-BamHI to isolate a 699 bp fragment. pNIV 4868 was digested with AflII-HpaI to isolate a 480 bp fragment. The two purified DNA fragments were inserted into plasmid pJW4304 previously cleaved with HpaI-BamHI. The resulting plasmids containing the ProDer p 1 variants C4R, C31R and C65R were called pNIV4879, pNIV4880 and pNIV4881.

### EXAMPLE XIII

Expression of ProDer p 1 in Pichia pastoris

1.—Construction of ProDer p 1 Expression Vector

The ProDer p 1 coding cassette from pNIV4846 (full-length 1-302aa ProDer p 1 cDNA with optimised mammalian codon usage) was amplified by PCR using the following primers: 5′ACTGACAGGCCTCGGCCGAGCTCCATTAA3′ (StuI restriction site in bold, forward) and 5′CAGTCACCTAGGTCTAGACTC GAGGGGAT3′ (AvrIl restriction site in bold, reverse). The amplified fragment was cloned into the pCR2.1 TOPO cloning vector. The correct ProDer p 1 cassette was verified by DNA sequencing. Recombinant TOPO vector was digested with StuI-AvrII to generate a 918 bp fragment which was introduced into the pPIC9K expression vector restricted with SnaBI-AvrII. The resulting plasmid, pNIV4878, contains the ProDer p 1 cassette downstream to the S.cerevisae α factor

2.—Site-Directed Mutagenesis

Expression plasmid for the production of unglycosylated ProDer p 1 (N52Q, mature Der p 1 numbering) was derived from pNIV4878 by overlap extension PCR using a set of four primers. The following primers:

5′GGCTTTCGAACACCTTAAGACCCAG3′ (primer 1, AflII restriction site in bold, forward) and 5′GCTCCCTAGCTACGTA TCGGTAATAGC3′ (primer 2, SnaBI restriction site in bold, reverse) were used to amplify a 317 bp fragment encoding the ProDer p 1 amino acid sequence 71-176.

The following primers 5′CCTCGCGTATCGGCAACAGAGCCTGGACC3′ (primer 3, mutation N52Q in bold, forward) and 5′GGTCCAGGCTCTGTTGCC GATACGCGAGG3′ (primer 4, mutation N52Q in bold, reverse) were used to introduce mutation N52Q in the ProDer p 1 sequence.

The mutated 317 bp AflII-SnaBI fragment was generated by a three-step process. In PCR n°1, primers 1 and 4 were mixed with pNIV4878 to produce a ˜200 bp fragment. In PCR n°2, primers 2 and 3 were mixed with pNIV4878 to produce a [140 bp. The two PCR products were purified onto agarose gel and used as templates for a third round of PCR to obtain a ˜340 bp fragment. This purified fragment was cloned into the pCR2.1 TOPO cloning vector (Invitrogen). The mutation was verified by DNA sequencing. Recombinant TOPO vector was digested with AflII-SnaBI to generate a 317 bp fragment which was ligated into the similarly digested pNIV4878. The resulting plasmid, pNIV4883, contains the ProDer p 1 N52Q downstream to the S.cerevisae α factor. To obtain unglycosylated variants of ProDer p 1 carrying mutations of Der p 1 cysteine residues at position 4, 31 or 65 (mature Der p 1 numbering), overlap extension PCR using the same set of primers were performed with plasmids pNIV4873, pNIV4875 and pNIV4874. The resulting plasmids pNIV4884, 4885 and 4886 encode respectively ProDer p 1 N52Q C4R, N52Q C31R and N52Q C65R.

2.—Transformation of P. pastoris

Plasmid pNIV4878 was introduced into P. pastoris using the spheroplast transformation method. Transformants were selected for histidinol deshydrogenase (His+) prototrophy. The screening of His+ transformants for geneticin (G418) resistance was performed by plating clones on agar containing increasing concentrations of G418.

Transformation with plasmids encoding ProDer p 1 N52Q, ProDer p 1 N52Q C4R, N52Q C31R and N52Q C65R was performed using the same method.

3.—Production of ProDer p 1 by Recombinant Yeast

G418 resistant clones were grown at 30° C. in BMG medium to an OD₆₀₀ₙₘ of 2-6. Cells were collected by centrifugation and resuspended to an OD₆₀₀ₙₘ of 1 in 100 ml of BMG medium. ProDer p 1 expression was induced by daily addition of methanol 0.5% for 6 days. The supernatant was collected by centrifugation and stored at −20° C. until purification.

4.—Purification of ProDer p 1 from Yeast Culture Supernatant

Supernatants were diluted 10 times with water and, after pH adjustment to 9, directly loaded onto a Q sepharose column equilibrated in in 20 mM Tris-HCl pH 9. The column was washed with the starting buffer. Protein elutions proceeded by step-wise increasing NaCl concentration in the buffer. The ProDer p 1-enriched fractions were pooled and concentrated by ultrafiltration onto a Filtron membrane (Omega serie, cut-off: 10 kD). The ProDer p 1 purification was achieved by a gel filtration chromatography onto a superdex-75 column (1×30 cm, Pharmacia) equilibrated in PBS pH 7,3. Purified ProDer p 1 was concentrated and stored at −20° C.

Sequence Information

| SEQ ID NO:1 | |
|---|---|
| 1 | CGGCCGAGCTGCATTAAGACCTTCGAGGAATACAAGAAAGCCTTCAAGAA |
| 51 | GAGCTATGCCACCTTCGAGGACGAGGAGGCCGGGCGCAAGAACTTCGTGG |
| 101 | AAAGCGTGAAATACGTGCAGAGCAACGGCGGGGCTATAAATCACCTGTCC |
| 151 | GACCTGTCTTTAGACGAGTTCAAGAACCGGTTGCTGATGAGCGCCGAGGC |
| 201 | TTTCGAACACCTTAAGACCCAGTTTGATCTCAACGCGGAGACCAACGCCT |
| 251 | GCAGTATCAACGGCAATGGCCCCGCTGAGATTGATCTGCGCCAGATGAGG |
| 301 | ACCGTGACTCCCATCCGCATGCAAGGCGGCTGCGGGTCTTGTTGGGCCTT |
| 351 | TTGAGGCGTGGCCGCGACAGAGTCGGCATACCTCGCGTATCGGAATCAGA |
| 401 | GCCTGGACCTCGCTGAGCAGGAGCTCGTTGACTGCGCCTCCCAACACGGA |
| 451 | TGTCATGGGGATACGATTCCCAGAGGTATCGAATACATCCAGCATAATGG |
| 501 | CGTCGTGCAGGAAAGCTATTACCGATACGTAGCTAGGGAGCAGTCCTGCC |
| 551 | GCCGTCCTAACGCACAGCGCTTCGGCATTTCCAATTATTGCCAGATCTAC |
| 601 | CCCCCTAATGCCAACAAGATCAGGGAGGCCCTGGCGCAGACGCACAGCGC |
| 651 | CATCGCTGTCATCATCGGAATCAAGGATCTGGACGCATTCCGGCACTATG |
| 701 | ACGGGCGCACAATCATCCAGCGCGACAACGGATATCAGCCAAACTACGAC |
| 751 | GCGGTCAACATCGTGGGTTACTCGAACGCCCAGGGOGTGGACTACTGGAT |
| 801 | CGTGAGAAACAGTTGGGACACTAACTGGGGCGACAACGOGTACGGCTACT |
| 851 | TCGCCGCCAACATCGACCTGATGATGATCGAGGAGTACCCGTACGTGGTG |
| 901 | ATCCTGTAA |

| SEQ ID NO:2 | |
|---|---|
| Arg Pro Ser Ser Ile Lys Thr Phe Gln Glu Tyr Lys Lys Ala Phe | 15 |
| Asn Lys Ser Tyr Ala Thr Phe Glu Asp Glu Glu Ala Ala Arg Lys | 30 |
| Asn Phe Leu Glu Ser Val Lys Tyr Val Gln Ser Asn Gly Gly Ala | 45 |
| Ile Asn His Leu Ser Asp Leu Ser Leu Asp Glu Phe Lys Asn Arg | 60 |
| Phe Leu Met Ser Ala Glu Ala Phe Glu His Leu Lys Tbr Gln Phe | 75 |
| Asp Leu Asn Ala Glu Tbr Asn Ala Cys Ser Ile Asn Gly Asn Ala | 90 |
| Pro Ala Glu Ile Asp Leu Aig Gln Met Arg Thr Val Tbr Pro Ile | 105 |
| Arg Met Gln Gly Gly Cys Gly Ser Cys Trp Ala Phe Ser Gly Val | 120 |
| Ala Ala Thr Glu Ser Ala Tyr Leu Ala Tyr Arg Asn Gln Ser Leu | 135 |
| Asp Leu Ala Glu Gln Glu Leu Val Asp Cys Ala Ser Gln His Gly | 150 |
| Cys His Gly Asp Thr Ile Pro Arg Gly Ile Glu Tyr Ile Gln His | 165 |
| Asn Gly Val Val Gln Giu Ser Tyr Tyr Arg Tyr Val Ala Arg Glu | 180 |
| Gln Ser Cys Arg Arg Pro Asn Ala Gln Arg Phe Gly Ile Ser Asn | 195 |
| Tyr Cys Gln Ile Tyr Pro Pro Asn Val Asn Lys Ile Arg Glu Ala | 210 |
| Leu Ala Gln Thr His Ser Ala Ile Ala Val Ile Ile Gly Ile Lys | 225 |
| Asp Leu Asp Ala Phe Arg His Tyr Asp Gly Arg Thr Ile Ile Gln | 240 |
| Arg Asp Asn Gly Tyr Gln Pro Asn Tyr His Ala Val Asn Ile Val | 255 |
| Gly Tyr Ser Asn Ala Gln Gly Val Asp Tyr Trp Ile Val Arg Asn | 270 |
| Ser Trp Asp Thr Asn Trp Gly Asp Asn Gly Tyr Gly Tyr Phe Ala | 285 |
| Ala Asn Ile Asp Leu Met Met Ile Glu Glu Tyr Pro Tyr Val Val | 300 |
| Ile Leu | 302 |
| SEQ ID NO:3 | |
| Arg Pro Ser Ser Ile Lys Thr Phe Glu Glu Tyr Lys Lys Ala Phe | 15 |
| Asn Lys Ser Tyr Ala Thr Phe Glu Asp Glu Glu Ala Ala Arg Lys | 30 |
| Asn Phe Leu Giu Ser Val Lys Tyr Val Gln Ser Asn Gly Gly Ala | 45 |
| Ile Asn His Leu Ser Asp Leu Ser Leu Asp Giu Phe Lys Asn Arg | 60 |
| Phe Leu Met Ser Ala Glu Ala Phe Glu His Leu Lys Thr Gln Phe | 75 |
| Asp Leu Asn Ala Glu Thr Asn Ala Arg Ser Ile Asn Gly Asn Ala | 90 |
| Pro Ala Glu Ile Asp Leu Arg Gln Met Arg Thr Val Thr Pro Ile | 105 |
| Arg Met Gln Gly Gly Cys Gly Ser Cys Trp Ala Phe Ser Gly Val | 120 |
| Ala Ala Thr Glu Ser Ala Tyr Leu Ala Tyr Arg Asn Gln Ser Leu | 135 |
| Asp Leu Ala Glu Gln Glu Leu Val Asp Cys Ala Ser Gln His Gly | 150 |
| Cys His Gly Asp Thr Ile Pro Arg Gly Ile Glu Tyr Ile Gln His | 165 |
| Asn Gly Val Val Gln Glu Ser Tyr Tyr Arg Tyr Val Ala Arg Glu | 180 |
| Gln Ser Cys Arg Arg Pro Asn Ala Gln Arg Phe Gly Ile Ser Asn | 195 |
| Tyr Cys Gln Ile Tyr Pro Pro Asn Val Asn Lys Ile Arg Glu Ala | 210 |
| Leu Ala Gln Thr His Ser Ala Ile Ala Val Ile Ile Gly Ile Lys | 225 |
| Asp Leu Asp Ala Phe Arg His Tyr Asp Gly Arg Thr Ile Ile Gln | 240 |
| Arg Asp Asn Gly Tyr Gln Pro Asn Tyr His Ala Val Asn Ile Val | 255 |
| Gly Tyr Ser Asn Ala Gln Gly Val Asp Tyr Trp Ile Val Arg Asn | 270 |
| Ser Trp Asp Thr Asn Trp Gly Asp Asn Gly Tyr Gly Tyr Phe Ala | 285 |
| Ala Asn Ile Asp Leu Met Met Ile Glu Glu Tyr Pro Tyr Val Val | 300 |
| Ile Leu | 302 |

| SEQ ID NO:4 | |
|---|---|
| 1 | CGGCCGAGCTCCATTAAGACCTTCGAGGAATACAAGAAAGCCTTCAACAA |
| 51 | GAGCTATGCCACCTTCGAGGACGAGGAGGCCGCGCGCAAGAACTTCCTGG |
| 101 | AAAGCGTGAAATACGTGCAGAGCAACGGCGGGGCTATAAATCACCTGTCC |
| 151 | GACCTGTCTTTAGACGAGTTCAAGAACCGGTTCCTGATGAGCGCCGAGGC |
| 201 | TTTCGAACACCTTAAGACCCAGTTTGATCTCAACGCGQAGACCAACGCCC |
| 251 | GTAGTATCAACGGCAATGCCCCCGCTGAGATTGATCTGCGCCAGATGAGG |
| 301 | ACCGTGACTCCCATCCGCATGCAAGGCGGCTGCGGGTCTTGTTGGGGCTT |
| 351 | TTCAGGCGTGGCCGCGACAGAGTCGGCATACCTCGCGTATCGGAATCAGA |
| 401 | GCCTGGACCTCGCTGAGCAGGAGCTCGTTGACTGCGGCTCCCAACAGGGA |
| 451 | TGTCATGGGGATACGATTCCCAGAGGTATCGAATACATCCAGCATAATGG |
| 501 | CGTCGTGCAGGAAAGCTATTACGGATACGTAGGTAGGGAGCAGTGCTGCC |
| 551 | GCGGTCGTAACGCACAGCGCTTCGGCATTTCCAATTATTGCCAGATCTAC |
| 601 | GCGCCTAATGCCAACAAGATCAGGGAGGCCCTGGCGCAGACGCACAGCGC |
| 651 | CATCGCTGTCATCATCGGAATCAAGGATCTGGACGCATTCCGGCACTATG |
| 701 | ACGGGCGCACAATCATCCAGCGCGACAAGGGATATCAGCGAAACTACCAC |
| 751 | GCGGTCAACATCGTGGGTTACTCGAACGCCCAGGGGGTGGACTACTGGAT |
| 801 | CGTGAGAAACAGTTGGGACACTAACTGGGGCGACAACGGCTACGGCTACT |
| 851 | TCGCCGCCAACATCGACCTGATGATGATCGAGGAGTACCCGTACGTGGTG |
| 901 | ATCCTGTAA |

| SEQ ID NO:5 | |
|---|---|
| Arg Pro Ser Ser Ile Lys Thr Phe Glu Glu Tyr Lys Lys Ala Phe | 15 |
| Asn Lys Ser Tyr Ala Thr Phe Glu Asp Glu Glu Ala Ala Arg Lys | 30 |
| Asn Phe Leu Glu Ser Val Lys Tyr Val Gln Ser Asn Gly Gly Ala | 45 |
| Ile Asn His Leu Ser Asp Leu Ser Leu Asp Glu Phe Lys Asn Arg | 60 |
| Phe Leu Met Ser Ala Glu Ala Phe Glu His Leu Lys Thr Gln Phe | 75 |
| Asp Leu Asn Ala Glu Thr Asn Ala Cys Ser Ile Asn Gly Asn Ala | 90 |
| Pro Ala Glu Ile Asp Leu Arg Gln Met Arg Thr Val Thr Pro Ile | 105 |
| Arg Met Gln Gly Gly Arg Gly Ser Cys Trp Ala Phe Ser Gly Val | 120 |
| Ala Ala Thr Glu Ser Ala Tyr Leu Ala Tyr Arg Asn Gln Ser Leu | 135 |
| Asp Leu Ala Glu Gln Glu Leu Val Asp Cys Ala Ser Gln His Gly | 150 |
| Gys His Gly Asp Thr Ile Pro Arg Gly Ile Glu Tyr Ile Gln His | 165 |
| Asn Gly Val Val Gln Glu Ser Tyr Tyr Arg Tyr Val Ala Arg Glu | 180 |
| Gln Ser Cys Arg Arg Pro Asn Ala Gln Arg Phe Gly Ile Ser Asn | 195 |
| Tyr Cys Gln Ile Tyr Pro Pro Asn Val Asn Lys Ile Arg Glu Ala | 210 |
| Lou Ala Gln Thr His Ser Ala Ile Ala Val Ile Ile Gly Ile Lys | 225 |
| Asp Leu Asp Ala Phe Arg His Tyr Asp Gly Arg Thr Ile Ile Gln | 240 |
| Arg Asp Asn Gly Tyr Gln Pro Asn Tyr His Ala Val Asn Ile Val | 255 |
| Gly Tyr Ser Asn Ala Gln Gly Val Asp Tyr Trp Ile Val Arg Asn | 270 |
| Ser Trp Asp Thr Asn Trp Gly Asp Asn Gly Tyr Gly Tyr Phe Ala | 285 |
| Ala Asn Ile Asp Lou Met Met Ile Glu Glu Tyr Pro Tyr Val Val | 300 |
| Ile Leu | 302 |

| SEQ ID NO:6 | |
|---|---|
| 1 | CGGCCGAGCTCCATTAAGACGTTCGAGGAATACAAGAAAGCCTTCAACAA |
| 51 | GAGGTATGCCACCTTCGAGGACGAGGAGGCCGCGCGOAAGAACTTCCTGG |
| 101 | AAAGCGTGAAATAGGTGCAGAGCAAGGGCGGGGCTATAAATCACCTGTCC |
| 151 | GACCTGTCTTTAGACGAGTTCAAGAACCGGTTCCTGATGAGCGCCGAGGG |
| 201 | TTTCGAACACCTTAAGACCCAGTTTGATCTCAACGCGGAGACCAACGCCT |
| 251 | GCAGTATCAACGGCAATGCCCCCGCTGAGATTGATCTGCGCCAGATGAGG |
| 301 | ACCGTGACTCCCATCCGCATGCAAGGCGGCCGTGGGTCTTGTTGGGCCTT |
| 351 | TTCAGGCGTGGCCGCGACAGAGTCGGCATACCTCGCGTATCGGAATCAGA |
| 401 | GCCTGGACCTCGCTGAGCAGGAGCTCGTTGACTGCGCCTCCCAACACGGA |
| 451 | TGTCATGGGGATACGATTCCCAGAGGTATCGAATACATCCAGCATAATGG |
| 501 | CGTCGTGCAGGAAAGCTATTACCGATACGTAGCTAGGGAGCAGTCCTGCC |
| 551 | GCCGTCCTAACGCACAGCGCTTCGGCATTTCCAATTATTGCCAGATCTAC |
| 601 | CCCCCTAATGCCAACAAGATCAGGGAGGCCCTGGCGGAGACGCACAGCGC |
| 651 | CATCGCTGTCATCATCGGAATCAAGGATCTGGACGCATTCCGGCACTATG |
| 701 | AGGGGCGCACAATCATCCAGCGCGACAACGGATATCAGCGAAACTACCAC |
| 751 | GCGGTCAACATCGTGGGTTACTCGAACGCCCAGGGGGTGGACTACTGGAT |
| 801 | CGTGAGAAACAGTTGGGACACTAACTGGGGCGACAACGGCTACGGCTACT |
| 851 | TCGCCGCCAACATCGACCTGATGATGATCGAGGAGTACCCGTACGTGGTG |
| 901 | ATCCTGTAA |

| SEQ ID NO:7 | |
|---|---|
| Arg Pro Ser Ser Ile Lys Thr Phe Glu Glu Tyr Lys Lys Ala Phe | 15 |
| Asn Lys Ser Tyr Ala Thr Phe Glu Asp Glu Glu Ala Ala Arg Lys | 30 |
| Asn Phe Leu Glu Ser Val Lys Tyr Val Gln Ser Asn Gly Gly Ala | 45 |
| Ile Asn His Leu Ser Asp Leu Ser Leu Asp Glu Phe Lys Asn Arg | 60 |
| Phe Leu Met Ser Ala Glu Ala Phe Glu His Leu Lys Thr Gln Phe | 75 |
| Asp Leu Asn Ala Glu Thr Asn Ala Cys Ser Ile Asn Gly Asn Ala | 90 |
| Pro Ala Glu Ile Asp Leu Arg Gln Met Arg Thr Val Thr Pro Ile | 105 |
| Arg Met Gln Gly Gly Cys Gly Ser Cys Trp Ala Phe Ser Gly Val | 120 |
| Ala Ala Thr Glu Ser Ala Tyr Leu Ala Tyr Arg Asn Gln Ser Leu | 135 |
| Asp Leu Ala Glu Gln Glu Leu Val Asp Arg Ala Ser Gln His Gly | 150 |
| Cys His Gly Asp Thr Ile Pro Arg Gly Ile Glu Tyr Ile Gln His | 165 |
| Asn Gly Val Val Gln Glu Ser Tyr Tyr Arg Tyr Val Ala Arg Glu | 180 |
| Gln Ser Cys Arg Arg Pro Asn Ala Gln Arg Phe Gly Ile Ser Asn | 195 |
| Tyr Cys Gln Ile Tyr Pro Pro Asn Val Asn Lys Ile Arg Glu Ala | 210 |
| Leu Ala Gln Thr His Ser Ala Ile Ala Val Ile Ile Gly Ile Lys | 225 |
| Asp Leu Asp Ala Phe Arg His Tyr Asp Gly Arg Thr Ile Ile Gln | 240 |
| Arg Asp Asn Gly Tyr Gln Pro Asn Tyr His Ala Val Asn Ile Val | 255 |
| Gly Tyr Ser Asn Ala Gln Gly Val Asp Tyr Trp Ile Val Arg Asn | 270 |
| Ser Trp Asp Thr Asn Trp Gly Asp Asn Gly Tyr Gly Tyr Phe Ala | 285 |
| Ala Asn Ile Asp Leu Met Met Ile Glu Glu Tyr Pro Tyr Val Val | 301 |
| Ile Leu | 302 |

| SEQ ID NO:8 | |
|---|---|
| 1 | CGGCCGAGCTCCATTAAGACCTTCGAGGAATACAAGAAAGCCTTCAACAA |
| 51 | GAGCTATGCCACCTTCGAGGACGAGGAGGCCGCGCGCAAGAACTTCCTGG |
| 101 | AAAGCGTGAAATACGTGCAGAGCAACGGCGGGGCTATAAATCACCTGTCC |
| 151 | GACCTGTCTTTAGACGAGTTCAAGAACCGGTTCCTGATGAGCGCCGAGGC |
| 201 | TTTCGAACAGCTTAAGACCCAGTTTGATCTGAACGCGGAGACCAACGCCT |
| 251 | GCAGTATCAACGGCAATGCCCCCGCTGAGATTGATCTGCGCCAGATGAGG |
| 301 | ACCGTGACTCCGATCCGCATGCAAGGCGGCTGCGGGTCTTGTTGGGCCTT |
| 351 | TTCAGGCGTGGCCGCGAGAGAGTCGGCATACCTCGCGTATCGGAATGAGA |
| 401 | GGCTGGACCTCGCTGAGCAGGAGCTCGTTGACCGTGCCTCCCAACACGGA |
| 451 | TGTGATGGGGATACGATTCGCAGAGGTATCGAATACATCCAGCATAATGG |
| 501 | CGTCGTGCAGGAAAGCTATTACCGATACGTAGCTAGGGAGCAGTCCTGCC |
| 551 | GCCGTCCTAACGCACAGCGCTTCGGCATTTCCAATTATTGCCAGATCTAC |
| 601 | CCCCCTAATGCCAACAAGATCAGGGAGGCCCTGGCGCAGACGCAGAGCGC |
| 651 | CATCGCTGTCATCATCGGAATCAAGGATCTGGACGCATTCCGGCACTATG |
| 701 | ACGGGCGCACAATCATCCAGCGCGACAACGGATATCAGCCAAACTACCAC |
| 751 | GCGGTCAACATGGTGCGTTACTCGAACGCCCAGGGGGTGGACTACTGGAT |
| 801 | CGTGAGAAACAGTTGGGACACTAACTGGGOCGACAACGGCTAGGGCTACT |
| 851 | TCGGCGCCAACATCGAGCTGATGATGATCGAGGAGTACCCGTACGTGGTG |
| 901 | ATCCTGTAA |

| SEQ ID NO:9 | |
|---|---|
| Arg Pro Ser Ser Ile Lys Thr Phe Glu Glu Tyr Lys Lys Ala Phe | 15 |
| Asn Lys Ser Tyr Ala Thr Phe Glu Asp Glu Glu Ala Ala Arg Lys | 30 |
| Asn Phe Leu Glu Ser Val Lys Tyr Val Gln Ser Asn Gly Gly Ala | 45 |
| Ile Asn His Leu Ser Asp Leu Ser Leu Asp Glu Phe Lys Asn Arg | 60 |
| Phe Leu Met Ser Ala Glu Ala Phe Glu His Leu Lys Thr Gln Phe | 75 |
| Asp Leu Asn Ala Glu Thr Asn Ala Cys Ser Ile Asn Gly Asn Ala | 90 |
| Pro Ala Glu Ile Asp Leu Arg Gln Met Arg Thr Val Thr Pro Ile | 105 |
| Arg Met Gln Gly Gly Cys Gly Ser Cys Trp Ala Phe Ser Gly Val | 120 |
| Ala Ala Thr Glu Ser Ala Tyr Leu Ala Tyr Arg Asn Gln Ser Leu | 135 |
| Asp Len Ala Gln Gln Gln Leu Val Asp Cys Ala Ser Gln His Gly | 150 |
| Arg His Gly Asp Thr Ile Pro Arg Gly Ile Glu Tyr Ile Gln His | 165 |
| Asn Gly Val Val Gln Gln Ser Tyr Tyr Arg Tyr Val Ala Arg Gln | 180 |
| Gln Ser Cys Arg Arg Pro Asn Ala Gln Arg Phe Gly Ile Ser Asn | 195 |
| Tyr Cys Gln Ile Tyr Pro Pro Asn Val Asn Lys Ile Arg Gln Ala | 210 |
| Len Ala Gln Thr His Ser Ala Ile Ala Val Ile Ile Gly Ile Lys | 225 |
| Asp Leu Asp Ala Phe Arg His Tyr Asp Gly Arg Thr Ile Ile Gln | 240 |
| Arg Asp Asn Gly Tyr Gln Pro Asn Tyr His Ala Val Asn Ile Val | 255 |
| Gly Tyr Ser Asn Ala Gln Gly Val Asp Tyr Trp Ile Val Arg Asn | 270 |
| Ser Trp Asp Thr Asn Trp Gly Asp Asn Gly Tyr Gly Tyr Phe Ala | 285 |
| Ala Asn Ile Asp Leu Met Met Ile Glu Gln Tyr Pro Tyr Val Val | 300 |
| Ile Leu | 302 |

| SEQ ID NO:10 | |
|---|---|
| 1 | CGGCCGAGCTCCATTAAGACCTTCGAGGAATACAAGAAAGCCTTCAACAA |
| 51 | GAGCTATGCCACCTTCGAGGACGAGGAGGCCGCGCGCAAGAACTTCCTGG |
| 101 | AAAGCGTGAAATACGTGCAGAGCAACGGCGGGGCTATAAATCACCTGTCC |
| 151 | GACCTGTCTTTAGACGAGTTCAAGAACCGGTTCCTGATGAGCGCCGAGGC |
| 201 | TTTCGAACACCTTAAGACCCAGTTTGATCTCAACGCGGAGACCAACGCCT |
| 251 | GCAGTATCAACGGCAATGCCCCCGCTGAGATTGATCTGCGCCAGATGAGG |
| 301 | ACCGTGACTCCCATCCGCATGCAAGGCGGCTGCGGGTCTTGTTGGGCCTT |
| 351 | TTCAGGCGTGGCCGCGACAGAGTCGGCATACCTCGCGTATGGGAATCAGA |
| 401 | GCCTGGACCTCGCTGAGCAGGAGCTCGTTGACTGCGCCTGCCAACACGGA |
| 451 | CGTCATGGGGATACGATTCCCAGAGGTATCGAATACATCCAGCATAATGG |
| 501 | CGTCGTGCAGGAAAGCTATTACCGATACGTAGCTAGGGAGCAGTCCTGCC |
| 551 | GCCGTGCTAAGGCACAGCGCTTCGGCATTTCCAATTATTGCCAGATCTAC |
| 601 | CCCCCTAATGCCAACAAGATCAGGGAGGCGCTGGCGCAGACGCAGAGCGC |
| 651 | CATCGCTGTCATCATCGGAATCAAGGATCTGGACGCATTCCGGCACTATG |
| 701 | ACGGGCGCACAATCATCCAGCGCGACAACGGATATCAGCCAAACTACCAC |
| 751 | GCGGTCAACATCGTGGGTTACTCGAACGCCCAGGGGGTGGACTACTGGAT |
| 801 | CGTGAGAAACAGTTGGGACACTAACTGGGGCGACAACGGCTACGGCTACT |
| 851 | TCGCCGCCAACATCGACCTGATGATGATCGAGGAGTACCCGTACGTGGTG |
| 901 | ATCCTGTAA |

| SEQ ID NO:11 | |
|---|---|
| Arg Pro Ser Ser Ile Lys Thr Phe Glu Glu Tyr Lys Lys Ala Phe | 15 |
| Asn Lys Ser Tyr Ala Thr Phe Glu Asp Glu Glu Ala Ala Arg Lys | 30 |
| Asn Phe Leu Glu Ser Val Lys Tyr Val Gln Ser Asn Gly Gly Ala | 45 |
| Ile Asn His Leu Ser Asp Leu Ser Leu Asp Glu Phe Lys Asn Arg | 60 |
| Phe Leu Met Ser Ala Glu Ala Phe Glu His Leu Lys Thr Gln Phe | 75 |
| Asp Leu Asn Ala Glu Thr Asn Ala Cys Ser Ile Asn Gly Asn Ala | 90 |
| Pro Ala Glu Ile Asp Leu Arg Gln Met Arg Thr Val Thr Pro Ile | 105 |
| Arg Met Gln Gly Gly Cys Gly Ser Cys Trp Ala Phe Ser Gly Val | 120 |
| Ala Ala Thr Glu Ser Ala Tyr Leu Ala Tyr Arg Asn Gln Ser Leu | 135 |
| Asp Leu Ala Glu Gln Glu Leu Val Asp Cys Ala Ser Gln His Gly | 150 |
| Cys His Gly Asp Thr Ile Pro Arg Gly Ile Glu Tyr Ile Gln His | 165 |
| Asn Gly Val Val Gln Glu Ser Tyr Tyr Arg Tyr Val Ala Arg Glu | 180 |
| Gln Ser Arg Arg Arg Pro Asn Ala Gln Arg Phe Gly Ile Ser Asn | 195 |
| Tyr Cys Gln Ile Tyr Pro Pro Asn Val Asn Lys Ile Arg Glu Ala | 210 |
| Leu Ala Gln Thr His Ser Ala Ile Ala Val Ile Ile Gly Ile Lys | 225 |
| Asp Leu Asp Ala Phe Arg His Tyr Asp Gly Arg Thr Ile Ile Gln | 240 |
| Arg Asp Asn Gly Tyr Gln Pro Asn Tyr His Ala Val Asn Ile Val | 255 |
| Gly Tyr Ser Asn Ala Gln Gly Val Asp Tyr Trp Ile Val Arg Asn | 270 |
| Ser Trp Asp Thr Asn Trp Gly Asp Asn Gly Tyr Gly Tyr Phe Ala | 285 |
| Ala Asn Ile Asp Leu Met Met Ile Glu Glu Tyr Pro Tyr Val Val | 300 |
| Ile Leu | 302 |

| SEQ ID NO:12 | |
|---|---|
| 1 | CGGCCGAGCTCCATTAAGACCTTCGAGGAATACAAGAAAGCCTTCAACAA |
| 51 | GAGCTATGCCACCTTCGAGGACGAGGAGGCCGCGCGCAAGAACTTCCTGG |
| 101 | AAAGCGTGAAATACGTGCAGAGCAACGGCGGGGCTATAAATCACCTGTCC |
| 151 | GACCTGTCTTTAGACGAGTTCAAGAACCGGTTCCTGATGAGCGCCGAGGC |
| 201 | TTTCGAACACCTTAAGACCCAGTTTGATCTCAACGCGGAGACCAACGCCT |
| 251 | GCAGTATCAACGACAATGCCCCCGCTGAGATTGATCTGCGCCAGATGAGG |
| 301 | ACCGTGACTCCCATCCGCATGCAAGGCGGCTGCGGGTCTTGTTGGGCCTT |
| 351 | TTCAGGCGTGGCCGCGACAGAGTCGGCATACCTCGCGTATCGGAATCAGA |
| 401 | GCCTGGACCTCGCTGAGCAGGAGCTCGTTGACTGCGCCTCCCAACACGGA |
| 451 | TGTCATGGGGATACGATTCGCAGAGGTATCGAATACATGCAGCATAATGG |
| 501 | CGTCGTGCAGGAAAGCTAtTACCGATACGTAGCTAGGGAGCAGTCCCGTC |
| 551 | GGGGTCCTAAGGCACAGCGCTTCGGCATTTCCAATTATTGCCAGATCTAC |
| 601 | CCCCCTAATGCCAACAAGATCAGGGAGGCCCTGGCGCAGACGCACAGCGG |
| 651 | CATCGCTGTCATCATCGGAATCAAGGATCTGGACGCATTGCGGCACTATG |
| 701 | ACGGGCGCACAATCATCCAGCGCGACAACGGATATCAGCCAAACTACCAC |
| 751 | GCGGTCAACATCGTGGGTTACTCGAAGGCCCAGGGGGTGGACTACTGGAT |
| 801 | CGTGAGAAACAGTTGGGACACTAACTGGGGCGACAACGGCTACGGCTACT |
| 851 | TCGCCGCCAACATCGACCTGATGATGATCGAGGAGTACCCGTACGTGGTG |
| 901 | ATCCTGTAA |

| SEQ ID NO:13 | |
|---|---|
| Arg Pro Ser Ser Ile Lys Thr Phe Glu Glu Tyr Lys Lys Ala Phe | 15 |
| Asn Lys Ser Tyr Ala Thr Phe Glu Asp Glu Glu Ala Ala Arg Lys | 30 |
| Asn Phe Leu Glu Ser Val Lys Tyr Val Gln Ser Asn Gly Gly Ala | 45 |
| Ile Asn His Leu Ser Asp Leu Ser Leu Asp Glu Phe Lys Asn Arg | 60 |
| Phe Leu Met Ser Ala Glu Ala Phe Glu His Leu Lys Thr Gln Phe | 75 |
| Asp Leu Asn Ala Glu Thr Asn Ala Cys Ser Ile Asn Gly Asn Ala | 90 |
| Pro Ala Glu Ile Asp Leu Arg Gln Met Arg Thr Val Thr Pro lIe | 105 |
| Arg Met Gln Gly Gly Cys Gly Ser Cys Trp Ala Phe Ser Gly Val | 120 |
| Ala Ala Thr Glu Ser Ala Tyr Leu Ala Tyr Arg Asn Gln Ser Leu | 135 |
| Asp Leu Ala Glu Gln Glu Leu Val Asp Cys Ala Ser Gln His Gly | 150 |
| Cys His Gly Asp Thr Ile Pro Arg Gly Ile Glu Tyr Ile Gln His | 165 |
| Asn Gly Val Val Gln Glu Ser Tyr Tyr Arg Tyr Val Ala Arg Glu | 180 |
| Gln Ser Cys Arg Arg Pro Asn Ala Gln Arg Phe Gly Ile Ser Asn | 195 |
| Tyr Arg Gln Ile Tyr Pro Pro Asn Val Asn Lys Ile Arg Glu Ala | 210 |
| Leu Ala Gln Thr His Ser Ala Ile Ala Val Ile Ile Gly Ile Lys | 225 |
| Asp Leu Asp Ala Phe Arg His Tyr Asn Gly Arg Thr Ile Ile Gln | 240 |
| Arg Asp Asn Gly Tyr Gln Pro Asn Tyr His Ala Val Asn Ile Val | 255 |
| Gly Tyr Ser Asn Ala Gln Gly Val Asp Tyr Trp Ile Val Arg Asn | 270 |
| Ser Trp Asp Thr Asn Trp Gly Asp Asn Gly Tyr Gly Tyr Phe Ala | 285 |
| Ala Asn Ile Asp Leu Met Met Ile Glu Glu Tyr Pro Tyr Val Val | 300 |
| Ile Leu | 302 |

| SEQ ID NO:14 | |
|---|---|
| 1 | CGGGCGAGCTCCATTAAGACCTTCGAGGAATACAAGAAAGCCTTCAACAA |
| 51 | GAGCTATGCCACCTTCGAGGACGAGGAGGCCGCGCGCAAGAAGTTCCTGG |
| 101 | AAAGCGTGAAATACGTGCAGAGCAACGGCGGGGCTATAAATCAGCTGTCC |
| 151 | GACCTGTCTTTAGACGAGTTCAAGAACCGGTTCCTGATGAGCGCCGAGGC |
| 201 | TTTCGAACACCTTAAGACCCAGTTTGATCTCAACGCGGAGACCAACGCCT |
| 251 | GCAGTATCAACGGCAATGCCCCCGCTGAGATTGATCTGCGCCAGATGAGG |
| 301 | ACCGTGACTGCCATCCGCATGCAAGGCGGCTGCGGGTCTTGTTGGGGCTT |
| 351 | TTCAGGCGTGGCCGCGACAGAGTCGGGATACCTCGCGTATCGGAATCAGA |
| 401 | GCCTGGACCTCGCTGAGCAGGAGCTCGTTGACTGCGCCTCCCAACACGGA |
| 451 | TGTCATGGGGATACGATTCCCAGAGGTATCGAATACATCCAGCATAATGG |
| 501 | CGTCGTGCAGGAAAGCTATTAGCGATACGTAGCTAGGGAGCAGTCCTGCG |
| 551 | GCCGTCCTAACGCACAGCGCTTCGGCATTTCGAATTATCGTCAGATCTAC |
| 601 | CGCCCTAATGCCAACAAGATCAGGGAGGCCCTGGCGCAGACGCACAGCGC |
| 651 | CATCGCTGTCATCATCGGAATCAAGGATCTGGACGCATTGCGGCACTATG |
| 701 | ACGGGCGCACAATCATCCAGCGCGACAACGGATATCAGCCAAACTACGAC |
| 751 | GCGGTCAACATCGTGGGTTACTCGAACGGCGAGGGGGTGGACTACTGGAT |
| 801 | GGTGAGAAACAGTTGGGACACTAACTGGGGCGACAACGGCTACGGCTACT |
| 851 | TCGCCGCCAACATCGACCTGATGATGATCGAGGAGTACCCGTACGTGGTG |
| 901 | ATCCTGTAA |

| SEQ ID NO:15 | |
|---|---|
| ProDerp 1 C71,103,117A (Der p 1 numbering) | |
| Arg Pro Ser Ser Ile Lys Thr Phe Glu Gln Tyr Lys Lys Ala Phe | 15 |
| Asn Lys Ser Tyr Ala Thr Phe Glu Asp Glu Glu Ala Ala Arg Lys | 30 |
| Asn Phe Leu Glu Set Val Lys Tyr Val Gln Set Asn Gly Gly Ala | 45 |
| Ile Asn His Leu Set Asp Leu Set Len Asp Glu Phe Lys Asn Arg | 60 |
| Phe Leu Met Set Ala Glu Ala Phe Glu His Leu Lys Thr Gln Phe | 75 |
| Asp Leu Asn Ala Gln Thr Asn Ala Cys Ser Ile Asn Gly Asn Ala | 90 |
| Pro Ala Glu Ile Asp Leu Arg Gln Met Arg Thr Val Thr Pro Ile | 105 |
| Arg Met Gln Gly Gly Cys Gly Set Cys Trp Ala Phe Ser Gly Val | 120 |
| Ala Ala Thr Glu Ser Ala Tyr Leu Ala Tyr Arg Asn Gln Ser Leu | 135 |
| Asp Len Ala Glu Gln Glu Leu Val Asp Cys Ala Set Gln His Gly | 150 |
| Ala His Gly Asp Thr Ile Pro Arg Gly Ile Glu Tyr Ile Gln His | 165 |
| Asn Gly Val Vat Gln Glu Set Tyr Tyr Arg Tyr Vat Ala Arg Gln | 180 |
| Gln Set Ala Arg Atg Pro Asn Ala Gln Arg Phe Gly Ile Ser Asn | 195 |
| Tyr Ala Gln Ile Tyr Pro Pro Asn Val Asn Lys Ile Arg Glu Ala | 210 |
| Leu Ala Gln Thr His Set Ala Ile Ala Val Ile Ile Gly Ile Lys | 225 |
| Asp Leu Asp Ala Phe Arg His Tyr Asp Gly Arg Thr Ile Ile Gln | 240 |
| Arg Asp Asn Gly Tyr Gln Pro Asn Tyr His Ala Vat Asn Ile Val | 255 |
| Gly Tyr Ser Asn Ala Gln Gly Val Asp Tyr Trp Ile Val Arg Asn | 270 |
| Ser Trp Asp Thr Asn Trp Gly Asp Asn Gly Tyr Gly Tyr Phe Ala | 285 |
| Ala Asn Ile Asp Leu Met Met Ile Glu Glu Tyr Pro Tyr Val Val | 300 |
| Ile Leu | 302 |

| SEQ ID NO:16 | |
|---|---|
| ProDer p 1 C71,103,117A (Der p 1 numbering) | |
| 1 | CGGCCGAGCTCCATTAAGACCTTCGAGGAATACAAGAAAGCCTTCAACAA |
| 51 | GAGCTATGCCACCTTCGAGGACGAGGAGGCCGCGCGCAAGAACTTCCTGG |
| 101 | AAAGCGTGAAATACGTGCAGAGCAACGGCGGGGCTATAAATCACCTGTCC |
| 151 | GACCTGTCTTTAGACGAGTTCAAGAACCGGTTCCTGATGAGCGCCGAGGC |
| 201 | TTTCGAACACCTTAAGACCCAGTTTGATCTCAACGCGGAGACCAACGCCT |
| 251 | GCAGTATCAACGGCAATGCCCCCGCTGAGATTGATCTGCGCCAGATGAGG |
| 301 | ACCGTGACTCCCATCCGCATGCAAGGCGGCTGCGGGTCTTGTTGGGCCTT |
| 351 | TTCAGGCGTGGCCGCGACAGAGTCGGCATACCTCGCGTATCGGAATCAGA |
| 401 | GCCTGCAGCTGGCTGAGCAGGAGCTCGTTGACTGCGCCTCCCAACACGGA |
| 451 | GCTCATGGGGATACGATTCCCAGAGGTATCGAATACATCCAGCATAATGG |
| 501 | CGTCGTGCAGGAAAGGTATTACCGATACGTAGCTAGGGAGCAGTCCGCCC |
| 551 | GCCGTGCTAACGCACAGCGCTTCGGCATTTCCAATTATGCCCAGATCTAC |
| 601 | CCCCCTAATGCCAACAAGATCAGGGAGGCCCTGGCGCAGACGCACAGCGG |
| 651 | CATCGCTGTCATCATCGGAATCAAGGATCTGGACGCATTGCGGCACTATG |
| 701 | ACGGGCGCACAATCATCCAGGGCGACAACGGATATCAGCCAAACTACCAG |
| 751 | GCGGTGAACATGGTGGGTTACTCGAACGCCCAGGGGGTGGACTACTGGAT |
| 801 | CGTGAGAAACAGTTGOGACACTAACTGGGGCGACAACGGCTACGGCTACT |
| 851 | TCGCCGGCAACATCGACGTGATGATGATCGAGGAGTACCCGTACGTGGTG |
| 901 | ATCCTGTAA |

| SEQ ID NO:17 | |
|---|---|
| ProDer p 1 delta 147-160 (Der p 1 numbering) | |
| Arg Pro Ser Ser Ile Lys Thr Phe Glu Glu Tyr Lys Lys Ala Phe | 15 |
| Asn Lys Ser Tyr Ala Thr Phe Glu Asp Glu Glu Ala Ala Arg Lys | 30 |
| Asn Phe Leu Glu Ser Val Lys Tyr Val Gln Ser Asn Gly Gly Ala | 45 |
| Ile Asn His Leu Ser Asp Leu Ser Leu Asp Glu Phe Lys Asn Arg | 60 |
| Phe Leu Met Ser Ala Glu Ala Phe Glu His Leu Lys Thr Gln Phe | 75 |
| Asp Leu Asn Ala Glu Thr Asn Ala Cys Ser Ile Asn Gly Asn Ala | 90 |
| Pro Ala Glu Ile Asp Leu Arg Gln Met Arg Thr Val Thr Pro Ile | 105 |
| Arg Met Gln Gly Gly Cys Gly Ser Cys Trp Ala Phe Ser Gly Val | 120 |
| Ala Ala Thr Glu Ser Ala Tyr Leu Ala Tyr Arg Asn Gln Ser Leu | 135 |
| Asp Leu Ala Glu Gln Glu Leu Val Asp Cys Ala Ser Gln His Gly | 150 |
| Cys His Gly Asp Thr Ile Pro Arg Gly Ile Glu Tyr Ile Gln His | 165 |
| Asn Gly Val Val Gln Glu Ser Tyr Tyr Arg Tyr Val Ala Arg Glu | 180 |
| Gln Ser Cys Arg Arg Pro Asn Ala Gln Arg Phe Gly Ile Ser Asn | 195 |
| Tyr Cys Gln Ile Tyr Pro Pro Asn Val Asn Lys Ile Arg Glu Ala | 210 |
| Leu Ala Gln Thr His Ser Ala Ile Ala Val Ile Ile Gly Ile Lys | 225 |
| Asp - - - - - - - - - - | 240 |
| Arg Asp Asn Gly Tyr Gln Pro Asn Tyr His Ala Val Asn Ile Val | 255 |
| Gly Tyr Ser Asn Ala Gln Gly Val Asp Tyr Trp Ile Val Arg Asn | 270 |
| Ser Trp Asp Thr Asn Trp Gly Asp Asn Gly Tyr Gly Tyr The Ala | 285 |
| Ala Asn Ile Asp Leu Met Met Ile Glu Glu Tyr Pro Tyr Val Val | 300 |
| Ile Leu | 302 |

SEQ ID NO:18
ProDer p 1 delta 147-160 (Der p 1 numbering)

| | |
|---|---|
| 1 | CGGCCGAGCTGCATTAAGACGTTCGAGGAATACAAGAAAGCCTTCAACAA |
| 51 | GAGCTATGCCACCTTCGAGGACGAGGAGGCCGCGCGCAAGAACTTCCTGG |
| 101 | AAAGCGTGAAATACGTGCAGAGCAACGGCGGGGCTATAAATCACCTGTCC |
| 151 | GACCTGTCTTTAGACGAGTTCAAGAACCGGTTCGTGATGAGCGCCGAGGC |
| 201 | TTTCGAACACCTTAAGACCCAGTTTGATCTCAACGCGGAGACCAACGCCT |
| 251 | GCAGTATCAAGGGCAATGCCCCCGCTGAGATTGATCTGCGCCAGATGAGG |
| 301 | ACCGTGACTCCCATCCGCATGCAAGGCGGCTGCGGGTCTTGTTGGGCCTT |
| 351 | TTCAGGCGTGGCCGCGACAGAGTCGGCATACCTCGCGTATCGGAATCAGA |
| 401 | GCCTGGACCTCGCTGAGCAGGAGCTCGTTGACTGCGCCTCCCAACACGGA |
| 451 | TGTCATGGGGATACGATTCCCAGAGGTATCGAATACATCCAGCATAATGG |
| 501 | CGTCGTGCAGGAAAGCTATTACCGATACGTAGCTAGGGAGCAGTCCTGCC |
| 551 | GCCGTCCTAACGCAGAGGGCTTCGGCATTTCCAATTATTGCCAGATCTAC |
| 601 | CCCCCTAATGCCAACAAGATCAGGGAGGCCCTGGCGGAGACGCACAGCGC |
| 651 | CATCGCTGTCATCATCGGAATCAAGGAT---------------------- |
| 701 | --------------------CGCGAGAACGGGTATCAGCCAAACTACCAC |
| 751 | GCGGTCAACATCGTGGGTTACTCGAACGCCCAGGGGGTGGACTACTGGAT |
| 801 | CGTGAGAAACAGTTGGGACACTAAGTGGGGCGACAACGGCTAGGGCTACT |
| 851 | TCGCCGCCAACATGGACCTGATGATGATCGAGGAGTAGCCGTACGTGGTG |
| 901 | ATCCTGTAA |

| SEQ ID NO: 19 | | |
|---|---|---|
| Amino acid sequence of PreProDer p 3 | | |
| Full sequence of PreProDer p 3 is amino-acids 1-261 | | |
| Prosequence is amino-acids 19-261 | | |
| Mature Der p 3 is amino-acids 30-261 | | |
| | 1 2 3 4 5 6 7 8 9 10 11 12 13 14 15 | |
| 1 | Met Ile Ile Tyr Asn Ile Leu Ile Val Leu Leu Leu Ala Ile Asn | 15 |
| 16 | Thr Leu Ala Asn Pro Ile Leu Pro Ala Ser Pro Asn Ala Thr Ile | 30 |
| 31 | Val Gly Gly Glu Lys Ala Leu Ala Gly Glu Cys Pro Tyr Gln Ile | 45 |
| 46 | Ser Leu Gln Ser Ser Ser His Phe Cys Gly Gly Thr Ile Leu Asp | 60 |
| 61 | Glu Tyr Trp Ile Leu Thr Ala Ala His Cys Val Ala Gly Gln Thr | 75 |
| 76 | Ala Ser Lys Leu Ser Ile Arg Tyr Asn Ser Leu Lys His Ser Leu | 90 |
| 91 | Gly Gly Glu Lys Ile Ser Val Ala Lys Ile Phe Ala His Glu Lys | 105 |
| 106 | Tyr Asp Ser Tyr Gln Ile Asp Asn Asp Ile Ala Leu Ile Lys Leu | 120 |
| 121 | Lys Ser Pro Met Lys Leu Asn Gln Lys Asn Ala Lys Ala Val Gly | 135 |
| 136 | Leu Pro Ala Lys Gly Ser Asp Val Lys Val Gly Asp Gln Val Arg | 150 |
| 151 | Val Ser Gly Trp Gly Tyr Leu Glu Glu Gly Ser Tyr Ser Leu Pro | 165 |
| 166 | Ser Glu Leu Arg Arg Val Asp Ile Ala Val Val Ser Arg Lys Glu | 180 |
| 181 | Cys Asn Glu Leu Tyr Ser Lys Ala Asn Ala Glu Val Thr Asp Asn | 195 |
| 196 | Met Ile Cys Gly Gly Asp Val Ala Asn Gly Gly Lys Asp Ser Cys | 210 |
| 211 | Gln Gly Asp Ser Gly Gly Pro Val Val Asp Val Lys Asn Asn Gln | 225 |
| 226 | Val Val Gly Ile Val Ser Trp Gly Tyr Gly Cys Ala Arg Lys Gly | 240 |
| 241 | Tyr Pro Gly Val Tyr Thr Arg Val Gly Asn Phe Ile Asp Trp Ile | 255 |
| 256 | Glu Ser Lys Arg Ser Gln | |

| SEQ ID NO: 20 | |
|---|---|
| Natural cDNA sequence of PreProDer p 3 | |
| cDNA of Leader peptide is 1-786 | |
| cDNA of Prosequence is 55-786 | |
| cDNA of Mature Der p 3 is 88-786 | |
| 1 | atgatcatct ataatatttt aattgtttta ttattggcca ttaatacatt ggctaatcca |
| 61 | attctaccag catcaccaaa tgcaactatt gttggtggtg aaaaagcatt agctggtgaa |
| 121 | tgtccatatc agatttcatt acaatcaagt agtcattttt gtggtggtac tattcttgat |
| 181 | gaatattgga ttttaacagc tgcacattgt gttgccggac aaacagcaag taaactttca |
| 241 | attcgttaca atagtttaaa acattcatta ggtggtgaaa aaatttctgt tgctaaaatt |
| 301 | tttgcacatg aaaaatatga tagttatcaa attgataatg atattgcatt gattaagctt |
| 361 | aaatcaccta tgaaattaaa tcagaaaaat gccaaagctg ttggattacc agcaaaagga |
| 421 | tcggatgtaa aagttggtga tcaagttcgt gtttctggtt ggggttatct tgaagaagga |
| 481 | agttattcat taccatctga attaagacgt gttgatattg ctgttgtatc acgtaaagaa |
| 541 | tgtaatgaat tatattcaaa agctaatgct gaagttactg ataatatgat ttgtggtggt |
| 601 | gatgttgcaa atggtggtaa agattcttgt caaggtgatt ctggtggacc ggttgttgat |
| 661 | gttaaaaata atcaagttgt tggtattgtt tcatggggtt atggttgtgc acgtaaaggt |
| 721 | tatccaggtg tttatacacg tgttggtaat tttatcgatt ggattgaatc aaaacgttca |
| 781 | cagtga |
| SEQ ID NO:21 | |
| Synthetic cDNA sequence of PreProDer p3 | |
| cDNA of Leader peptide is 1-786 | |
| cDNA of Prosequence is 55-786 | |
| cDNA of Mature Der p 3 is 88-786 | |
| The modified nucleotides are in bold and underlined | |
| 1 | atgatcatct aCaaCattCt GatCgtACtC CtGCtggcca ttaaCacTtt ggctaatccG |
| 61 | atCctGccGg catcCccGaa CgcGacCatC gttggCggCg aaaaagcACt GgctggtgaG |
| 121 | tgCccatatc agatCtcCCt GcaGtcTagt agCcaCttCt gtggtggtac tattcttgaC |
| 181 | gaataCtgga tCCtGacCgc G gcacaCtgCg tGgccggCc aaacagcGag CaaactCtcC |
| 241 | attcgttaca aCagCCtGaa acaCtcaCtG ggtggCgaaa aGatttctgt tgctaaaatt |
| 301 | ttCgcacatg aaaaatatga tagCtaCcaG atCgaCaatg aCattgcGCt gatCaagctG |
| 361 | aaatcCccta tgaaGCtGaa CcagaaaaaC gccaaagctg tGggCCtGcc GgcGaaaggC |
| 421 | tcggatgtaa aagttggtga CcaGgtGcgt gtCtctggCt ggggttatct GgaagaGggC |
| 481 | agCtaCtcCC tGccGtctga attaCg+E,un Ccgt gttgatatCg gtgtGgtatc TcgCaaagaa |
| 541 | tgtaaCgaGc tGtaCtcGaa agcGaaCgct gaagtCacCg aCaatatgat CtgCggtggt |
| 601 | gatgttgc+E,un Ga aCggCggtaa GgaCtcttgt caaggCgatt ctggtggGcc ggtGgtCgaC |
| 661 | gttaaaaaCa aCcaGgttgt AggtatCgtt tcAtggggCt aCggttgCgc acgtaaaggC |
| 721 | tatccGggtg tGtaGacCcg CgttggtaaC tttatcgatt ggattgaatc TaaacgtAGC |
| 781 | cagtga |

1. A recombinant Dermatophagoides pteronyssinus Der p 1 protein allergen derivative wherein said allergen derivative has a significantly reduced allergenic activity compared to that the wild-type allergen in which said derivative has been genetically mutated and in which the mutant comprises the three following mutations: a mutation of the cysteine 71 residue, a mutation of the cysteine 103 residue and a mutation of the cysteine 117 residue.

2. A recombinant Dermatophagoides pteronyssinus Der p 1 protein allergen derivative wherein said allergen derivative has a significantly reduced allergenic activity compared to that the wild-type allergen in which said derivative has been genetically mutated and in which the mutant comprises a deletion of amino acid residues 147 to 160 of Der p 1 or residues 227-140 of ProDer p 1.

3. A recombinant mutant allergen having any of the sequences selected from the group consisting of:, SEQ ID NO: 15, SEQ ID NO: 17.

4. An isolated nucleic acid molecule encoding a mutated version of an allergen as claimed in claim 1.

5. A nucleic acid sequence according to claim 4 wherein the codon usage pattern resembles that of highly expressed mammalian genes.

6. An expression vector containing a nucleic acid of claim 4.

7. A host cell transformed with a nucleic acid sequence of claim 4 or with a vector as claimed in claim 6.

8. An immunogenic composition comprising a recombinant protein or mutant allergen as claimed in claim 1, or an encoding polynucleotide as claimed in claim 4, and, optionally, an adjuvant.

9. An immunogenic composition as claimed in claim 8, wherein the adjuvant is a preferential stimulator of Th1-type immune responses.

10. An immunogenic composition as claimed in claim 8 wherein the adjuvant comprises one or more of 3D-MPL, QS21, a CpG oligonucleotide, a polyethylene ether or ester or a combination of two or more of these adjuvants.

11. An immunogenic composition as claimed in claim 8 wherein the allergen is presented in an oil in water or a water in oil emulsion vehicle.

12. A immunogenic composition as claimed herein for use in medicine.

13. Use of a recombinant protein or mutant allergen as claimed in claim 1 in the manufacture of a medicament for the treatment of allergy.

14. A method of treating a patient suffering from or preventing a patient susceptible to allergic responses, comprising administering to said individual an immunogenic composition as claimed in claim 8.

15. A Dermatophagoides pteronyssinus ProDer p 3 or PreProDer p 3 protein allergen or derivative thereof, wherein said ProDer p 3, PreProDer p 3 or allergen derivative has a significantly reduced allergenic activity compared to Der p 3.

16. An allergen or derivative as claimed in claim 15, wherein said allergen or derivative has been thermally treated.

17. A allergen or derivative as claimed in claim 15, wherein said allergen or derivative has been genetically mutated.

18. A allergen or derivative as claimed in claim 17, wherein the mutation comprises a mutation of a cysteine residue.

19. A recombinant allergen having the sequence of: SEQ ID NO:19.

20. An isolated nucleic acid molecule encoding a mutated version of an allergen as claimed in claim 1.

21. An isolated nucleic acid molecule having the sequence of any of SEQ ID NOs.20 or 21.

22. An expression vector containing a nucleic acid of claim 20.

23. A host cell transformed with a nucleic acid sequence of claim 20 or with a vector as claimed in claim 22.

24. An immunogenic composition comprising a recombinant protein or mutant allergen as claimed in claim 15, or an encoding polynucleotide as claimed in claim 20, and, optionally, an adjuvant.

25. An immunogenic composition as claimed in claim 24, wherein the adjuvant is a preferential stimulator of Th1-type immune responses.

26. An immunogenic composition as claimed in claim 24 wherein the adjuvant comprises one or more of 3D-MPL, QS21, a CpG oligonucleotide, a polyethylene ether or ester or a combination of two or more of these adjuvants.

27. An immunogenic composition as claimed in claim 24 wherein the allergen is presented in an oil in water or a water in oil emulsion vehicle.

28. A immunogenic composition as claimed herein for use in medicine.

29. Use of a recombinant protein or mutant allergen as claimed in claim 15 in the manufacture of a medicament for the treatment of allergy.

30. A method of treating a patient suffering from or preventing a patient susceptible to allergic responses, comprising administering to said individual an immunogenic composition as claimed in claim 24.


SEQUENCE LISTING (26 sequences)

SEQ ID NO: 1 - DNA, 909 bp, Dermatophagoides pteronyssinus
  CDS (1)...(906)
cggccgagctccattaagaccttcgaggaatacaagaaagccttcaacaagagctatgccaccttcgaggacgaggaggccgcgcgcaagaacttcctggaaagcgtgaaatacgtgcagagcaacggcggggctataaatcacctgtccgacctgtctttagacgagttcaagaaccggttcctgatgagcgccgaggctttcgaacaccttaagacccagtttgatctcaacgcggagaccaacgcctgcagtatcaacggcaatgcccccgctgagattgatctgcgccagatgaggaccgtgactcccatccgcatgcaaggcggctgcgggtcttgttgggccttttcaggcgtggccgcgacagagtcggcatacctcgcgtatcggaatcagagcctggacctcgctgagcaggagctcgttgactgcgcctcccaacacggatgtcatggggatacgattcccagaggtatcgaatacatccagcataatggcgtcgtgcaggaaagctattaccgatacgtagctagggagcagtcctgccgccgtcctaacgcacagcgcttcggcatttccaattattgccagatctacccccctaatgccaacaagatcagggaggccctggcgcagacgcacagcgccatcgctgtcatcatcggaatcaaggatctggacgcattccggcactatgacgggcgcacaatcatccagcgcgacaacggatatcagccaaactaccacgcggtcaacatcgtgggttactcgaacgcccagggggtggactactggatcgtgagaaacagttgggacactaactggggcgacaacggctacggctacttcgccgccaacatcgacctgatgatgatcgaggagtacccgtacgtggtgatcctgtaa

SEQ ID NO: 2 - protein, 302 aa, Dermatophagoides pteronyssinus
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 3 - protein, 302 aa, Artificial Sequence
  C4R mutant of ProDer p 1
RPSSIKTFEEYKKAFNKSYATFEDEEAARKNFLESVKYVQSNGGAINHLSDLSLDEFKNRFLMSAEAFEHLKTQFDLNAETNARSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 4 - DNA, 909 bp, Artificial Sequence
  CDS (1)...(906): C4R mutant of ProDer p 1
cggccgagctccattaagaccttcgaggaatacaagaaagccttcaacaagagctatgccaccttcgaggacgaggaggccgcgcgcaagaacttcctggaaagcgtgaaatacgtgcagagcaacggcggggctataaatcacctgtccgacctgtctttagacgagttcaagaaccggttcctgatgagcgccgaggctttcgaacaccttaagacccagtttgatctcaacgcggagaccaacgcccgtagtatcaacggcaatgcccccgctgagattgatctgcgccagatgaggaccgtgactcccatccgcatgcaaggcggctgcgggtcttgttgggccttttcaggcgtggccgcgacagagtcggcatacctcgcgtatcggaatcagagcctggacctcgctgagcaggagctcgttgactgcgcctcccaacacggatgtcatggggatacgattcccagaggtatcgaatacatccagcataatggcgtcgtgcaggaaagctattaccgatacgtagctagggagcagtcctgccgccgtcctaacgcacagcgcttcggcatttccaattattgccagatctacccccctaatgccaacaagatcagggaggccctggcgcagacgcacagcgccatcgctgtcatcatcggaatcaaggatctggacgcattccggcactatgacgggcgcacaatcatccagcgcgacaacggatatcagccaaactaccacgcggtcaacatcgtgggttactcgaacgcccagggggtggactactggatcgtgagaaacagttgggacactaactggggcgacaacggctacggctacttcgccgccaacatcgacctgatgatgatcgaggagtacccgtacgtggtgatcctgtaa

SEQ ID NO: 5 - protein, 302 aa, Artificial Sequence
  C31R mutant of ProDer p 1
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGRGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 6 - DNA, 909 bp, Artificial Sequence
  CDS (1)...(906): C31R mutant of ProDer p 1
cggccgagctccattaagaccttcgaggaatacaagaaagccttcaacaagagctatgccaccttcgaggacgaggaggccgcgcgcaagaacttcctggaaagcgtgaaatacgtgcagagcaacggcggggctataaatcacctgtccgacctgtctttagacgagttcaagaaccggttcctgatgagcgccgaggctttcgaacaccttaagacccagtttgatctcaacgcggagaccaacgcctgcagtatcaacggcaatgcccccgctgagattgatctgcgccagatgaggaccgtgactcccatccgcatgcaaggcggccgtgggtcttgttgggccttttcaggcgtggccgcgacagagtcggcatacctcgcgtatcggaatcagagcctggacctcgctgagcaggagctcgttgactgcgcctcccaacacggatgtcatggggatacgattcccagaggtatcgaatacatccagcataatggcgtcgtgcaggaaagctattaccgatacgtagctagggagcagtcctgccgccgtcctaacgcacagcgcttcggcatttccaattattgccagatctacccccctaatgccaacaagatcagggaggccctggcgcagacgcacagcgccatcgctgtcatcatcggaatcaaggatctggacgcattccggcactatgacgggcgcacaatcatccagcgcgacaacggatatcagccaaactaccacgcggtcaacatcgtgggttactcgaacgcccagggggtggactactggatcgtgagaaacagttgggacactaactggggcgacaacggctacggctacttcgccgccaacatcgacctgatgatgatcgaggagtacccgtacgtggtgatcctgtaa

SEQ ID NO: 7 - protein, 302 aa, Artificial Sequence
  C65R mutant of ProDer p 1
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDRASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 8 - DNA, 909 bp, Artificial Sequence
  CDS (1)...(906): C65R mutant of ProDer p 1
cggccgagctccattaagaccttcgaggaatacaagaaagccttcaacaagagctatgccaccttcgaggacgaggaggccgcgcgcaagaacttcctggaaagcgtgaaatacgtgcagagcaacggcggggctataaatcacctgtccgacctgtctttagacgagttcaagaaccggttcctgatgagcgccgaggctttcgaacaccttaagacccagtttgatctcaacgcggagaccaacgcctgcagtatcaacggcaatgcccccgctgagattgatctgcgccagatgaggaccgtgactcccatccgcatgcaaggcggctgcgggtcttgttgggccttttcaggcgtggccgcgacagagtcggcatacctcgcgtatcggaatcagagcctggacctcgctgagcaggagctcgttgaccgtgcctcccaacacggatgtcatggggatacgattcccagaggtatcgaatacatccagcataatggcgtcgtgcaggaaagctattaccgatacgtagctagggagcagtcctgccgccgtcctaacgcacagcgcttcggcatttccaattattgccagatctacccccctaatgccaacaagatcagggaggccctggcgcagacgcacagcgccatcgctgtcatcatcggaatcaaggatctggacgcattccggcactatgacgggcgcacaatcatccagcgcgacaacggatatcagccaaactaccacgcggtcaacatcgtgggttactcgaacgcccagggggtggactactggatcgtgagaaacagttgggacactaactggggcgacaacggctacggctacttcgccgccaacatcgacctgatgatgatcgaggagtacccgtacgtggtgatcctgtaa

SEQ ID NO: 9 - protein, 302 aa, Artificial Sequence
  C71R mutant of ProDer p 1
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGRHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 10 - DNA, 909 bp, Artificial Sequence
  CDS (1)...(906): C71R mutant of ProDer p 1
cggccgagctccattaagaccttcgaggaatacaagaaagccttcaacaagagctatgccaccttcgaggacgaggaggccgcgcgcaagaacttcctggaaagcgtgaaatacgtgcagagcaacggcggggctataaatcacctgtccgacctgtctttagacgagttcaagaaccggttcctgatgagcgccgaggctttcgaacaccttaagacccagtttgatctcaacgcggagaccaacgcctgcagtatcaacggcaatgcccccgctgagattgatctgcgccagatgaggaccgtgactcccatccgcatgcaaggcggctgcgggtcttgttgggccttttcaggcgtggccgcgacagagtcggcatacctcgcgtatcggaatcagagcctggacctcgctgagcaggagctcgttgactgcgcctcccaacacggacgtcatggggatacgattcccagaggtatcgaatacatccagcataatggcgtcgtgcaggaaagctattaccgatacgtagctagggagcagtcctgccgccgtcctaacgcacagcgcttcggcatttccaattattgccagatctacccccctaatgccaacaagatcagggaggccctggcgcagacgcacagcgccatcgctgtcatcatcggaatcaaggatctggacgcattccggcactatgacgggcgcacaatcatccagcgcgacaacggatatcagccaaactaccacgcggtcaacatcgtgggttactcgaacgcccagggggtggactactggatcgtgagaaacagttgggacactaactggggcgacaacggctacggctacttcgccgccaacatcgacctgatgatgatcgaggagtacccgtacgtggtgatcctgtaa

SEQ ID NO: 11 - protein, 302 aa, Artificial Sequence
  C103R mutant of ProDer p 1
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSRRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 12 - DNA, 909 bp, Artificial Sequence
  CDS (1)...(906): C103R mutant of ProDer p 1
cggccgagctccattaagaccttcgaggaatacaagaaagccttcaacaagagctatgccaccttcgaggacgaggaggccgcgcgcaagaacttcctggaaagcgtgaaatacgtgcagagcaacggcggggctataaatcacctgtccgacctgtctttagacgagttcaagaaccggttcctgatgagcgccgaggctttcgaacaccttaagacccagtttgatctcaacgcggagaccaacgcctgcagtatcaacggcaatgcccccgctgagattgatctgcgccagatgaggaccgtgactcccatccgcatgcaaggcggctgcgggtcttgttgggccttttcaggcgtggccgcgacagagtcggcatacctcgcgtatcggaatcagagcctggacctcgctgagcaggagctcgttgactgcgcctcccaacacggatgtcatggggatacgattcccagaggtatcgaatacatccagcataatggcgtcgtgcaggaaagctattaccgatacgtagctagggagcagtcccgtcgccgtcctaacgcacagcgcttcggcatttccaattattgccagatctacccccctaatgccaacaagatcagggaggccctggcgcagacgcacagcgccatcgctgtcatcatcggaatcaaggatctggacgcattccggcactatgacgggcgcacaatcatccagcgcgacaacggatatcagccaaactaccacgcggtcaacatcgtgggttactcgaacgcccagggggtggactactggatcgtgagaaacagttgggacactaactggggcgacaacggctacggctacttcgccgccaacatcgacctgatgatgatcgaggagtacccgtacgtggtgatcctgtaa

SEQ ID NO: 13 - protein, 302 aa, Artificial Sequence
  C117R mutant of ProDer p 1
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYRQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 14 - DNA, 909 bp, Artificial Sequence
  CDS (1)...(906): C117R mutant of ProDer p 1
cggccgagctccattaagaccttcgaggaatacaagaaagccttcaacaagagctatgccaccttcgaggacgaggaggccgcgcgcaagaacttcctggaaagcgtgaaatacgtgcagagcaacggcggggctataaatcacctgtccgacctgtctttagacgagttcaagaaccggttcctgatgagcgccgaggctttcgaacaccttaagacccagtttgatctcaacgcggagaccaacgcctgcagtatcaacggcaatgcccccgctgagattgatctgcgccagatgaggaccgtgactcccatccgcatgcaaggcggctgcgggtcttgttgggccttttcaggcgtggccgcgacagagtcggcatacctcgcgtatcggaatcagagcctggacctcgctgagcaggagctcgttgactgcgcctcccaacacggatgtcatggggatacgattcccagaggtatcgaatacatccagcataatggcgtcgtgcaggaaagctattaccgatacgtagctagggagcagtcctgccgccgtcctaacgcacagcgcttcggcatttccaattatcgtcagatctacccccctaatgccaacaagatcagggaggccctggcgcagacgcacagcgccatcgctgtcatcatcggaatcaaggatctggacgcattccggcactatgacgggcgcacaatcatccagcgcgacaacggatatcagccaaactaccacgcggtcaacatcgtgggttactcgaacgcccagggggtggactactggatcgtgagaaacagttgggacactaactggggcgacaacggctacggctacttcgccgccaacatcgacctgatgatgatcgaggagtacccgtacgtggtgatcctgtaa

SEQ ID NO: 15 - DNA, 108 bp, Artificial Sequence
  Primer
ttaagacccagtttgatctcaacgcggagaccaacgcccgtatcaacggcaatgcccccgctgagattgatctgcgccagatgaggaccgtgactcccatccgcatgc

SEQ ID NO: 16 - DNA, 103 bp, Artificial Sequence
  Primer
cggatgggagtcacggtcctcatctggcgcagatcaatctcagcgggggcattgccgttgatactacgggcgttggtctccgcgttgagatcgaaactgggtc

SEQ ID NO: 17 - DNA, 92 bp, Artificial Sequence
  Primer
caaggcggccgtgggtcttgttgggccttttcaggcgtggccgcgacagagtcggcatacctcgcgtatcggaatcagagcctggacctcgc

SEQ ID NO: 18 - DNA, 99 bp, Artificial Sequence
  Primer
tcagcgaggtccaggctctgattccgatacgcgaggtatgccgactctgtcgcggccacgcctgaaaaggcccaacaagacccacggccgccttgcatg

SEQ ID NO: 19 - DNA, 83 bp, Artificial Sequence
  Primer
tgagcaggagctcgttgaccgtgcctcccaacacggatgtcatggggatacgattcccagaggtatcgaatacatccagcata

SEQ ID NO: 20 - DNA, 77 bp, Artificial Sequence
  Primer
ctggatgtattcgatacctctgggaatcgtatcccccatgacatccgtgttgggaggcacggtcaacgcgctcctgc

SEQ ID NO: 21 - DNA, 29 bp, Artificial Sequence
  Primer
actgacaggcctcggccgagctccattaa

SEQ ID NO: 22 - DNA, 29 bp, Artificial Sequence
  Primer
cagtcacctaggtctagactcgaggggat

SEQ ID NO: 23 - DNA, 25 bp, Artificial Sequence
  Primer
ggctttcgaacaccttaagacccag

SEQ ID NO: 24 - DNA, 27 bp, Artificial Sequence
  Primer
gctccctagctacgtatcggtaatagc

SEQ ID NO: 25 - DNA, 29 bp, Artificial Sequence
  Primer
cctcgcgtatcggcaacagagcctggacc

SEQ ID NO: 26 - DNA, 29 bp, Artificial Sequence
  Primer
ggtccaggctctgttgccgatacgcgagg